SEQUENCE LISTING (32 sequences)

SEQ ID NO: 1 - DNA, 3205 bp, Homo sapiens
  CDS (110)..(2428)
  ref <308>: DATABASE ACCESSION NUMBER: NCBI/NM_015478
  ref <309>: DATABASE ENTRY DATE: 2013-02-17
  ref <313>: RELEVANT RESIDUES IN SEQ ID NO: (1)..(3205)
accccgcaggtgcttgggggcgtagcctggagtgagggtttggctggtgtagcttggagtgaggccccctggcgtggagtcttgaggcctgctgagggcgtgagctggcatgaggcgaagagagggccatggcaccgactccgagatgggtcaaggacccgtacgggagtcgcaatcctcagaccctcccgcgctccagttccggataagcgagtataagccgctgaacatggcgggagtggagcagcccccgagccccgagctgcggcaggaaggcgtgaccgaatacgaagatggcggggccccggcgggagatggcgaggcgggcccccaacaggcggaggaccacccccagaatcctccagaagatcccaatcaggaccccccagaggatgatagcacctgtcagtgccaggcgtgcgggcctcaccaagccgcgggtccagatcttggttcctctaatgatggctgccctcagctgttccaggagcggtcagtcatagtggagaactcctcaggctctaccagcgcttctgagctcctcaaacccatgaagaagaggaagcgcagggaataccagagcccatcagaggaggagtcggagccagaggccatggagaagcaagaagaaggaaaggacccagagggacaacccactgctagcaccccagagagtgaggagtggagcagcagccagcctgcaacaggtgagaagaaggaatgctggtcgtgggagtcctacctagaggagcagaaggccattactgctccagtcagcctcttccaggactcccaggcagtcactcacaacaagaatggcttcaaactgggcatgaagttggaaggcattgaccctcaacacccgtccatgtacttcatcctcaccgtggctgaggtatgtggctatcgcctacgcctgcactttgatgggtattctgagtgccatgacttctgggtcaatgccaactcccctgacattcaccctgctggctggttcgagaagacgggccacaagctgcagcctcccaaaggttacaaggaggaggagttcagctggagccagtacctgcgcagcacaagagctcaggctgcccccaagcacctgtttgtgagccagagccacagtcccccacccctgggcttccaggtgggcatgaagctggaggctgttgaccgcatgaacccgtcccttgtctgcgtggccagtgtgaccgatgtggtggacagccgcttcctggtgcactttgacaactgggatgatacttatgactactggtgtgatcccagcagcccctacatccacccagtgggctggtgccagaagcaaggaaagcccctcacccctccacaagactacccagaccctgataacttctgttgggagaaatatctggaagaaactggggcctctgctgtccccacctgggccttcaaggtgcgaccccctcacagcttcctggtcaatatgaagctggaggctgtggaccgcaggaacccagccctgattcgcgtggccagcgtggaggatgtggaggaccatcggataaagatccactttgatggctggagtcatggctatgatttctggatcgacgctgaccacccagacatccaccctgccggctggtgctccaagacaggacatcccctgcagcctcctctcggacccagagagcccagctctgcctcccctgggggctgtccccctctcagctataggagcctgccccacactaggacctccaaatacagctttcaccaccggaagtgccccactcctggttgcgacggctctggccatgtcacaggcaagttcacagctcaccattgcctctcaggctgcccactggctgagaggaaccagagccggctgaaagcggagctgtctgactcggaggcctcagcccgcaagaagaacctctcaggcttctccccaaggaagaagcctcgccatcacggccgaattggacgccctccgaagtatcgaaagattccgcaggaagatttccagaccctcacgcccgatgtcgtgcaccagtccctcttcatgtcagccctgtcggcccaccctgaccgctcactctcagtgtgctgggagcagcactgcaagctcctgccaggagtagcgggcatctcagcctcgacagtcgccaagtggaccatcgatgaggtcttcggctttgttcagaccctgacaggttgtgaggaccaagcacgcctcttcaaagacgaggcaagaatagtcagagtgacccatgtatctgggaagactctagtctggactgtggcccagcttggggaccttgtgtgctcagatcatcttcaggaaggaaaaggcatcctggagacaggagtccattcactcctctgctctctacccactcatttgcttgccaaacttagctttgccagtgatagtcaatattaaagtgtacttttttcccctttaatccaatatagttgataattaaagtgtattttgaatgacacagatattgtgatttactgcaaggatcctaacacacacttaaaatcaagagccaaggagtagtgagttgtagataaaaaaagaatgtcagctttggagacagtctgggtttaaatcccagttctgtcaatttgagctgtttactgtctctgagcctacatcttcttgtctgtaaaatggagataaaatgggtttaatgaggtctaccttgcagagccattgtgagcattggaaatgatgaatgaatcataccagaacgtctagtataattacagtcatgcattgcttaacgatggggatacattcttagaaatgtgtcactaggcaattctgtcattgtgtaaacattatagaatgtacttacacaaacctagatgttatatgtatttttatttacatgtatattttcacatgaaataccaaatgtcacagcattattactgaatgtcagtcatttcccctacttgatctgcaatgccaatatcaagggccatgtatcaggtttctgtatatgttccactataatcttatgggaccatggttttaaatgtggaatcattgacagaaatgtctttatgtagcatatggctgtgtatcactagtatataatagagcaatattatggaggaatatgtagatccaatcactttacctatacaaaatgactgctatggtgggaacacaataaacaccagttttgacttttaaaaaaaaaaa

SEQ ID NO: 2 - protein, 772 aa, Homo sapiens
MRRREGHGTDSEMGQGPVRESQSSDPPALQFRISEYKPLNMAGVEQPPSPELRQEGVTEYEDGGAPAGDGEAGPQQAEDHPQNPPEDPNQDPPEDDSTCQCQACGPHQAAGPDLGSSNDGCPQLFQERSVIVENSSGSTSASELLKPMKKRKRREYQSPSEEESEPEAMEKQEEGKDPEGQPTASTPESEEWSSSQPATGEKKECWSWESYLEEQKAITAPVSLFQDSQAVTHNKNGFKLGMKLEGIDPQHPSMYFILTVAEVCGYRLRLHFDGYSECHDFWVNANSPDIHPAGWFEKTGHKLQPPKGYKEEEFSWSQYLRSTRAQAAPKHLFVSQSHSPPPLGFQVGMKLEAVDRMNPSLVCVASVTDVVDSRFLVHFDNWDDTYDYWCDPSSPYIHPVGWCQKQGKPLTPPQDYPDPDNFCWEKYLEETGASAVPTWAFKVRPPHSFLVNMKLEAVDRRNPALIRVASVEDVEDHRIKIHFDGWSHGYDFWIDADHPDIHPAGWCSKTGHPLQPPLGPREPSSASPGGCPPLSYRSLPHTRTSKYSFHHRKCPTPGCDGSGHVTGKFTAHHCLSGCPLAERNQSRLKAELSDSEASARKKNLSGFSPRKKPRHHGRIGRPPKYRKIPQEDFQTLTPDVVHQSLFMSALSAHPDRSLSVCWEQHCKLLPGVAGISASTVAKWTIDEVFGFVQTLTGCEDQARLFKDEARIVRVTHVSGKTLVWTVAQLGDLVCSDHLQEGKGILETGVHSLLCSLPTHLLAKLSFASDSQY

SEQ ID NO: 3 - protein, 16 aa, Homo sapiens
VGLSGAPADACSTAQK

SEQ ID NO: 4 - protein, 11 aa, Homo sapiens
NQALFPACVLK

SEQ ID NO: 5 - protein, 11 aa, Homo sapiens
GSFKYAWVLDK

SEQ ID NO: 6 - protein, 12 aa, Homo sapiens
MDSTEPPYSQKR

SEQ ID NO: 7 - protein, 14 aa, Homo sapiens
ETVSEESNVLCLSK

SEQ ID NO: 8 - protein, 13 aa, Homo sapiens
SILSPGGSCGPIK

SEQ ID NO: 9 - protein, 10 aa, Homo sapiens
FGEVVDCTLK

SEQ ID NO: 10 - protein, 36 aa, Homo sapiens
IIPTLEEGLQLPSPTATSQLPLESDAVECLNYQHYK

SEQ ID NO: 11 - protein, 21 aa, Homo sapiens
YGGFLLASYDEDSEELQAICK

SEQ ID NO: 12 - protein, 20 aa, Homo sapiens
LGEINVIGEPFLNVNCEHIK

SEQ ID NO: 13 - protein, 29 aa, Homo sapiens
DSVSGELTLEGGALVLADQGVCCIDEFDK

SEQ ID NO: 14 - protein, 25 aa, Homo sapiens
TFFGDDFIPNDILIGCEEEEQENGK

SEQ ID NO: 15 - protein, 12 aa, Homo sapiens
LLDLVQQSCNYK

SEQ ID NO: 16 - protein, 22 aa, Homo sapiens
AKENDENCGPTTTVFVGNISEK

SEQ ID NO: 17 - protein, 21 aa, Homo sapiens
GNLYSFGCPEYGQLGHNSDGK

SEQ ID NO: 18 - protein, 10 aa, Homo sapiens
ILDILGETCK

SEQ ID NO: 19 - protein, 15 aa, Homo sapiens
LVDSPDIQPVGTCEK

SEQ ID NO: 20 - protein, 30 aa, Homo sapiens
FGGQLLSFDIGDQPVFEIPLSNVSQCTTGK

SEQ ID NO: 21 - protein, 17 aa, Homo sapiens
EIVHLQAGQCGNQIGAK

SEQ ID NO: 22 - protein, 8 aa, Homo sapiens
NVSVKDVR

SEQ ID NO: 23 - protein, 11 aa, Homo sapiens
EIAQDFKTDLR

SEQ ID NO: 24 - protein, 12 aa, Homo sapiens
AQYKGAASEAGR

SEQ ID NO: 25 - protein, 13 aa, Homo sapiens
SLLEQAQKPIDIR

SEQ ID NO: 26 - protein, 11 aa, Homo sapiens
AIKSPPGFSAK

SEQ ID NO: 27 - protein, 28 aa, Homo sapiens
SGDAAIVDMVPGKPMCVESFSDYPPLGR

SEQ ID NO: 28 - protein, 8 aa, Homo sapiens
LLEMLQSK

SEQ ID NO: 29 - protein, 341 aa, Homo sapiens
EEWSSSQPATGEKKECWSWESYLEEQKAITAPVSLFQDSQAVTHNKNGFKLGMKLEGIDPQHPSMYFILTVAEVCGYRLRLHFDGYSECHDFWVNANSPDIHPAGWFEKTGHKLQPPKGYKEEEFSWSQYLRSTRAQAAPKHLFVSQSHSPPPLGFQVGMKLEAVDRMNPSLVCVASVTDVVDSRFLVHFDNWDDTYDYWCDPSSPYIHPVGWCQKQGKPLTPPQDYPDPDNFCWEKYLEETGASAVPTWAFKVRPPHSFLVNMKLEAVDRRNPALIRVASVEDVEDHRIKIHFDGWSHGYDFWIDADHPDIHPAGWCSKTGHPLQPPLGPREPSSASPGG

SEQ ID NO: 30 - protein, 4 aa, Artificial Sequence
  linker
GGSG

SEQ ID NO: 31 - protein, 4 aa, Artificial Sequence
  linker
SAGG

SEQ ID NO: 32 - protein, 5 aa, Artificial Sequence
  linker
GGGGS

(19) United States
(12) Patent Application Publication
(10) Pub. No.: US 2014/0274782 A1
(43) Pub. Date: Sep. 18, 2014

(54) AFFINITY REAGENTS AND METHODS FOR DETECTION, PURIFICATION, AND PROTEOMIC ANALYSIS OF METHYLATED PROTEINS

(71) Applicants: Scott M. Carlson, Menlo Park, CA (US); Or Gozani, Stanford, CA (US); Kaitlyn E. Moore, Mountain View, CA (US)

(72) Inventors: Scott M. Carlson, Menlo Park, CA (US); Or Gozani, Stanford, CA (US); Kaitlyn E. Moore, Mountain View, CA (US)

(73) Assignee: The Board of Trustees of the Leland Stanford Junior University, Palo Alto, CA (US)

(21) Appl. No.: 14/214,730

(22) Filed: Mar. 15, 2014

Related U.S. Application Data
(60) Provisional application No. 61/794,167, filed on Mar. 15, 2013.

(51) Int. Cl. G01N 33/68; G01N 33/573; C07K 14/47

(52) U.S. Cl. CPC G01N 33/6842; C07K 14/47; G01N 33/573
USPC 506/9; 530/350; 435/188; 530/402; 530/411; 436/501; 435/7.4; 435/7.92; 506/18; 536/23.5; 435/320.1; 435/252.33; 435/252.31; 435/252.3; 435/252.35; 435/254.21; 435/325; 435/358; 435/367; 435/352; 435/365; 435/370; 435/364; 435/254.2; 435/254.22; 435/254.23; 435/348; 435/69.1

(57) ABSTRACT

Methyl-lysine affinity reagents created by engineering the 3×MBT methyl-lysine binding domain repeat of lethal (3) malignant brain tumor-like protein 1 (L3MBTL1) are disclosed. In particular, the invention relates to affinity reagents and affinity chromatography media comprising the 3×MBT domain repeat and methods of using such affinity reagents in detection, purification, and proteomic profiling of methylated proteins and peptides.

### CROSS-REFERENCE TO RELATED APPLICATION

This application claims benefit under 35 U.S.C. §119(e) of provisional application 61/794,167, filed Mar. 15, 2013, which is hereby incorporated by reference in its entirety.

### STATEMENT REGARDING FEDERALLY SPONSORED RESEARCH OR DEVELOPMENT

This invention was made with Government support under contract GM079641 awarded by the National Institutes of Health. The Government has certain rights in this invention.

### TECHNICAL FIELD

The present invention pertains generally to affinity reagents and affinity-based techniques. In particular, the invention relates to methyl-lysine affinity reagents, containing the three malignant brain tumor domain repeat (3×MBT) of lethal (3) malignant brain tumor-like protein 1 (L3MBTL1), and methods of using such affinity reagents for detection, purification, and proteomic analysis of methylated proteins and peptides.

### BACKGROUND

Post-translational modification of histone and non-histone proteins by the addition of one, two, or three methyl groups to the 8-nitrogen of lysine residues is proposed to play important roles in key signal transduction pathways (Bannister and Kouzarides (2011) Cell Res. 21, 381-395; Greer and Shi (2012) Nat. Rev. Genet. 13, 343-357; Huang and Berger (2008) Curr. Opin. Genet. Dev. 18, 152-158; Margueron and Reinberg (2010) Nat. Rev. Genet. 11, 285-296). Besides histone proteins, where lysine methylation has been extensively studied, only a relatively small number of other proteins are known to be modified by lysine methylation (Huang and Berger, supra; Su and Tarakhovsky (2006) Curr. Opin. Immunol. 18, 152-157). However, as there are greater than 50 potential lysine methyltransferases (KMTs) and approximately 25 lysine demethylases (KDMTs) in the human genome (Greer and Shi, supra; Kooistra and Helin (2012) Nat. Rev. Mol. Cell Biol. 13, 297-311; Petrossian and Clarke (2011) Proteomics 10, M110.000976), it is highly likely that regulation of hundreds or thousands of proteins by lysine methylation remains to be discovered.

A common strategy for proteome-wide identification of post-translationally modified proteins has relied on the availability of modification-specific antibodies with no dependence on surrounding residues (“pan-specific”). Such an approach has been successful for modifications including phosphorylation, acetylation, and arginine methylation (Choudhary et al. (2009) Science 325, 834-840; Ong and Mann (2006) Curr. Protoc. Protein. Sci. Chapter 14, Unit 14.19; Zhang et al. (2005) Mol. Cell Proteomics 4, 1240-1250). In contrast, this type of strategy has not been possible for lysine methylation due to the absence of truly pan-specific antibodies. In addition, the ability to investigate newly discovered lysine methylation events has been limited by the difficulty and cost of raising modification-specific antibodies.

Thus, there remains a need for an affinity reagent that can selectively bind to methylated proteins, which can be used for detection, purification, and proteomic profiling of methylated proteins.

### SUMMARY

The invention relates to methyl-lysine affinity reagents created by engineering the 3×MBT methyl-lysine binding domain of L3MBTL1 and methods of using such affinity reagents in detection, enrichment, purification, and proteomic profiling of methylated proteins and peptides.

In one aspect, the invention includes an affinity reagent that binds to a methylated lysine residue, the affinity reagent comprising one or more engineered 3×MBT domains of one or more L3MBTL1 proteins, wherein each 3×MBT domain consists of the residues of a L3MBTL1 protein corresponding to amino acids 190 to 530 of human L3MBTL1, numbered relative to the reference sequence of SEQ ID NO:2. In certain embodiments, the affinity reagent comprises at least two 3×MBT domains. The 3×MBT domains in the affinity reagent can be the same or different.

In one embodiment, the affinity reagent comprises at least one 3×MBT domain comprising the amino acid sequence of SEQ ID NO:29 or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, wherein the 3×MBT domain is capable of binding to a protein or peptide comprising a mono-methylated or di-methylated lysine.

In certain embodiments, the affinity reagent further comprises a tag. Tags that can be used in the practice of the invention include, but are not limited to a glutathione S-transferase tag, a FLAG tag, a His-tag, a Strep-tag, a TAP-tag, an S-tag, an SBP-tag, an Arg-tag, a calmodulin-binding peptide tag, a cellulose-binding domain tag, a DsbA tag, a c-myc tag, a HAT-tag, a maltose-binding protein tag, a NusA tag, a biotin tag, and a thioredoxin tag.

In certain embodiments, the affinity reagent further comprises a detectable label, such as, but not limited to a radioactive isotope, a stable non-radioactive heavy isotope, a fluorophore, a chemiluminescer, an enzyme, and a ligand.

In certain embodiments, the affinity reagent further comprises one or more linkers connecting polypeptides (e.g., 3×MBT domains and/or tags and/or labels) within the affinity reagent or connecting an affinity reagent to a solid support. Linkers are typically short peptide sequences of 2-30 amino acid residues, often composed of glycine and/or serine residues. Linker sequences that can be used in the practice of the invention include, but are not limited to [Gly]ₓ, [Gly-Ser]ₓ, [Gly-Gly-Ser-Gly]ₓ (SEQ ID NO:30), [Ser-Ala-Gly-Gly]ₓ (SEQ ID NO:31), and [Gly-Gly-Gly-Gly-Ser]ₓ (SEQ ID NO:32), wherein x=1-15, and GSAT, SEG, and Z-EGFR linkers.

In certain embodiments, the affinity reagent is immobilized on a solid support. Exemplary solid supports include a bead (e.g., agarose bead, polystyrene bead, microbead, magnetic bead), a particle, a rod, a membrane (e.g., a nitrocellulose membrane or a polyvinylidene difluoride (PVDF) membrane), a gel, a resin, a column, a chip, a slide, a plate (e.g., a microtiter plate), and a microarray.

In one embodiment, the invention includes an affinity chromatography matrix comprising a methyl-lysine affinity reagent, described herein, bound to a solid support. Exemplary solid supports may comprise polyether sulfone, agarose, cellulose, a polysaccharide, polytetrafluoroethylene, polysulfone, polyester, polyvinylidene fluoride, polypropylene, poly (tetrafluoroethylene-co-perfluoro(alkyl vinyl ether)), polycarbonate, polyethylene, glass, polyacrylate, polyacrylamide, poly(azolactone), polystyrene, polylactide, ceramic, nylon or metal. The affinity chromatography matrix may further comprise a linking group, such as, but not limited to, cyanogen bromide, tresyl, triazine, vinyl sulfone, an aldehyde, an epoxide, or an activated carboxylic acid to facilitate coupling of the affinity agent to the solid support. Additionally, the affinity reagent may be connected to the solid support through a linker to make the affinity reagent more accessible for binding to methylated proteins and peptides.

In the practice of the invention, the methyl-lysine affinity reagents can be used in various methods, for example, in far-Western blotting, pull-down assays, or affinity chromatography, for detection, identification, enrichment, purification, or proteomic profiling of methylated proteins or peptides.

In one embodiment, the invention includes a method for of isolating a methylated protein or peptide from a mixture, the method comprising binding a methyl-lysine affinity reagent, described herein, to the methylated protein or peptide and performing a pull-down assay to isolate the methylated protein or peptide from the mixture.

In another embodiment, the invention includes a method for detecting methylation of a protein or peptide, the method comprising binding a methyl-lysine affinity reagent, described herein, to a methylated protein or peptide and detecting the bound affinity reagent. For example, the methylated protein or peptide can be detected by performing a far-western or a pull-down assay. In one embodiment, the affinity reagent comprises a detectable label wherein the bound affinity reagent is identified by detecting the label. In another embodiment, the affinity reagent comprises an epitope tag, wherein the bound affinity reagent is identified with a detectably labeled antibody that specifically binds to the epitope tag.

In another embodiment, the invention includes a method of measuring the activity of a lysine methyltransferase with a methyl-lysine affinity reagent described herein, the method comprising: a) contacting the lysine methyltransferase with a protein or peptide substrate comprising an unmethylated lysine; and b) detecting methylation of the lysine in the product of the enzyme catalyzed reaction by binding the affinity reagent to the methylated product. In one embodiment, the method further comprises identifying substrates of the lysine methyltransferase by contacting the lysine methyltransferase with a protein array comprising candidate protein or peptide substrates and determining which of the candidate proteins or peptides are methylated by detecting lysine methylation with an affinity reagent described herein. Both lysine mono-methyltransferases and di-methyltransferases may be screened by this method.

In another embodiment, the invention includes a method of isolating a methylated protein or peptide, the method comprising binding a methyl-lysine affinity reagent comprising an epitope tag to the methylated protein or peptide and immunoprecipitating the methylated protein or peptide with an antibody specific for the epitope tag.

In another embodiment, the invention includes a method of purifying a methylated protein or peptide from a mixture using an affinity chromatography matrix comprising bound methyl-lysine affinity reagent, as described herein, the method comprising: a) contacting the mixture with the affinity chromatography matrix under a first set of conditions, such that the methylated protein or peptide binds to the immobilized affinity reagent attached to the matrix; and b) eluting the methylated protein or peptide under a second set of conditions thereby purifying the methylated protein or peptide from the mixture. Affinity chromatography may be performed in a column or in batch.

In another embodiment, the invention includes a method of making an affinity chromatography matrix, the method comprising: a) activating a solid support; and b) contacting the solid support with a methyl-lysine affinity reagent, described herein, such that the affinity reagent covalently attaches to the solid support.

In another aspect, the invention includes a polynucleotide encoding an affinity reagent described herein. In one embodiment, the polynucleotide is a recombinant polynucleotide comprising a polynucleotide encoding an affinity reagent operably linked to a promoter. In certain embodiments, the recombinant polynucleotide comprises a polynucleotide encoding a polypeptide comprising the sequence of SEQ ID NO:29, or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, wherein the encoded affinity reagent comprises a 3×MBT domain and is capable of binding to a protein or peptide comprising a mono-methylated or di-methylated lysine. In certain embodiments, the recombinant polynucleotide comprises the contiguous sequence from nucleotide position 677 to nucleotide position 1697 of SEQ ID NO:1, or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, wherein the encoded affinity reagent comprises a 3×MBT domain and is capable of binding to a protein or peptide comprising a mono-methylated or di-methylated lysine.

In another aspect, the invention includes a host cell comprising a recombinant polynucleotide encoding an affinity reagent.

In another aspect, the invention includes a method for producing a methyl-lysine affinity reagent, the method comprising the steps of: a) culturing a host cell comprising a recombinant polynucleotide encoding an affinity reagent under conditions suitable for the expression of the affinity reagent; and b) recovering the affinity reagent from the host cell culture.

In another aspect, the invention includes a kit for preparing or using affinity reagents according to the methods described herein. Such kits may comprise one or more methyl-lysine affinity reagents, affinity chromatography matrices comprising affinity reagents, or reagents for preparing affinity reagents (e.g., expression vector encoding an affinity reagent, cells, solid support, affinity chromatography resin, activating agent, column, or other reagents for preparing affinity reagents), as described herein. Kits may further comprise reagents for performing a far Western, a pull-down assay, or affinity chromatography.

These and other embodiments of the subject invention will readily occur to those of skill in the art in view of the disclosure herein.

### BRIEF DESCRIPTION OF THE FIGURES

FIGS. 1A-1E show that 3×MBT recognizes methylated lysine with broad sequence specificity. FIG. 1A shows Coomassie stain of glutathione S-transferase (GST) alone, GST-tagged 3×MBT, and GST-tagged 3×MBT_{D355N}. FIG. 1B shows that 3×MBT, but not methyllysine-binding mutant 3×MBT_{D355N}, binds to mono- and di-methylated H4K20- and p53K382-containing peptides. A Western blot analysis is shown of peptide pull-downs with GST-3×MBT and GST-3×MBT_{D355N} and the indicated biotinylated peptides: H4 peptides: amino acids 1-23 and p53 peptides: amino acids 367-388. FIG. 1C shows that 3×MBT coprecipitates with a panel of biotinylated mono- and di-methylated peptides with varying sequences. Peptides are labeled by their methylated residues; all peptides are roughly 20 amino acids in length, with only the indicated residues methylated. FIG. 1D shows that 3×MBT does not bind to a series of non-methylated peptides. Pull-down experiments are shown with the indicated peptides as in FIG. 1C. FIG. 1E shows that 3×MBT binds preferentially to diverse mono- and di-methylated peptides. Microarrays spotted with over 100 distinct histone and non-histone peptides as indicated in the array key on the right were probed with GST-3×MBT. Spots indicate positive binding. 3×MBT does not bind unmodified peptides, tri-methylated peptides, or peptides bearing other post-translational modifications. *H3K79 peptides have low solubility and therefore are not transferred efficiently to the array surface.

FIGS. 2A-2E show that 3×MBT recognizes mono- and di-methyl lysine in the presence of secondary posttranslational modifications. FIGS. 2A-2E show a Western blot analysis of peptide pull-down assays with GST-3×MBT and GST-3×MBT_{D355N} and the indicated biotinylated peptides. All modified peptides have the same base sequence and length as the unmodified peptide control. FIG. 2A shows that phosphorylation of H3S10 modestly decreases binding of 3×MBT to mono- and di-methyl H3K9. FIG. 2B shows that phosphorylation of RelA at S311 decreases, but does not abolish, binding of 3×MBT to RelAK310me1. FIG. 2C shows that symmetric dimethylation of H3R2 does not block 3×MBT binding to H3K4me2-containing peptides. FIG. 2D shows that different states of methylation of H3R8 have little effect on binding of 3×MBT to H3K9me1- and H3K9me2-containing peptides. FIG. 2E shows that 3×MBT binds p53K372me2 in the presence of adjacent serine phosphorylation or lysine acetylation.

FIGS. 3A-3F show that 3×MBT detects multiple methylated proteins in common affinity-based assays. FIGS. 3A and 3B show that 3×MBT, but not 3×MBT_{D355N}, recognizes endogenous histones in Far Western assays. A Far Western analysis is shown of (FIG. 3A) purified HeLa nucleosomes and (FIG. 3B) purified calf thymus histones serially diluted to the indicated quantities and probed with GST-3×MBT and GST-3×MBT_{D355N}. Binding of GST fusion proteins was detected with α-GST antibody. *H3 indicates an H3 degradation product that migrates near H4. H3 blots are shown as a loading control. FIG. 3C shows that 3×MBT, but not 3×MBT_{D355N}, specifically detects monomethylated p53 by Far Western. In vitro methylation reactions with wild-type SET8 or catalytically inactive SET8_{D338A} on recombinant wild-type p53 or p53 carrying K382R substitution (p53_{K382R}) were probed with α-p53K382me1 antibody as previously described (Shi et al. (2007) Mol. Cell 27, 636-646) or the indicated GST fusion as in FIG. 3A. The cofactor SAM was included in the reactions as indicated. A Coomassie stain of p53 is shown below as a loading control. FIG. 3D shows that 3×MBT, but not 3×MBT_{D355N}, specifically detects recombinant H3 dimethylated at lysine 9 (H3K9me2). In vitro methylation reactions of recombinant H3 with recombinant G9a SET domain were detected as described in FIG. 3C. Total H3 is shown as a loading control. FIG. 3E shows that 3×MBT, but not 3×MBT_{D355N}, precipitates known methylated proteins H3 and p53 from HeLa nuclear extract (NE). Western blot of GST-3×MBT and GST-3×MBT_{D355N} NE pull-downs probed with the indicated antibodies. FIG. 3F shows that 3×MBT, but not 3×MBT_{D355N}, precipitates methylated RelA from cells. Pull-down assays are shown of NE from 293T cells co-expressing SETD6 (or two different negative control constructs, the second of which is indicated by the asterisks) and wild-type RelA or RelA carrying K310R substitution (RelA_{K310R}) and probed with the indicated antibodies. α-RelAK310me1 antibody was previously described (Levy et al. (2011) Nat. Immunol. 12, 29-36).

FIGS. 4A-4E show that 3×MBT detects a novel methylation event on the lysine deacetylase SIRT1 and on candidate G9a substrates. FIG. 4A shows a Coomassie stain of full-length recombinant GST-tagged SIRT1 and SIRT1_{K622R} purified from Sf9 cells.

FIG. 4B shows that G9a methylates SIRT1 as lysine 622. An autoradiograph is shown of in vitro methylation assays on SIRT1 and SIRT1_{K622R} using recombinant G9a SET domain. FIG. 4C shows that 3×MBT, but not 3×MBT_{D355N}, precipitates methylated SIRT1 from cells. GST3×MBT or GST-3×MBT_{D355N} pull-down assays were performed (as in FIG. 2E) of NE from 293T cells expressing 3× Flag-SIRT1 or 3× Flag-SIRT1_{K622R} and G9a or control vector. Pull-downs were probed with anti-Flag antibody to detect SIRT1. FIG. 4D shows that 3×MBT detects G9a-methylated substrates on protein microarrays. Representative blocks of human Invitrogen ProtoArrays were methylated in vitro using GST alone (left) or GST-tagged recombinant G9a SET domain (right). Methylation was detected by probing the arrays with 3× Flag-3×MBT, followed by α-Flag antibody and species-matched fluorescent secondary antibody. Magnified regions show examples of G9amethylated proteins detected by Flag-3×MBT. A list of all candidate G9a-methylated proteins is shown in Table 3. FIG. 4E shows a scatter plot comparing GST array signal-to-noise ratio (SNR) and G9a SNR for ranked ProtoArray hits shown in Table 3.

FIGS. 5A-5E show that protein pull-down using 3×MBT reproducibly enriches for the methyl-lysine proteome. FIG. 5A shows a protocol schematic for proteome-wide capture and identification of candidate methylated proteins using 3×MBT. Peptides derived from specifically enriched proteins show a distinctive signal in the stable isotope labeling by amino acids in cell culture (SILAC) associated with 3×MBT, while peptides from nonspecifically bound proteins (equal binding to 3×MBT and 3×MBT_{D355N} resins) have the same intensity in both SILAC conditions. FIG. 5B shows that abrogating the methyl-lysine binding site of 3×MBT greatly reduces the amount of protein captured from nuclear extract. Silver stain of total proteins present is shown for GST-3×MBT and GST-3×MBT_{D355N} pull-downs from 293T nuclear extracts. Asterisk indicates GST fusion band. FIG. 5C shows that specific proteins are reproducibly and quantitatively enriched by 3×MBT relative to 3×MBT_{D355N}. Axes represent the quantitative ratio (log base 2) for 3×MBT over 3×MBT_{D355N} in independent experiments. Each point represents a protein identified in both experiments, and enrichment in these experiments is correlated with R²=0.86. FIG. 5D shows that most proteins identified following enrichment by 3×MBT (approximately 60%) are specifically enriched by the methyl-lysine binding domain. Probability density plots show 3×MBT enrichment for all 544 identified proteins identified in pull-down experiments compared to the SILAC ratios of proteins identified from input material. FIG. 5E shows that proteins enriched by 3×MBT tend to be functionally related based on protein interaction networks. The diagram shows all high-confidence protein-protein interactions from the STRING interaction database between proteins enriched to at least 2-fold by 3×MBT relative to 3×MBT_{D355N}.

FIGS. 6A-6D show candidate physiologic substrates of G9a and GLP are identified by targeted inhibition and quantitative proteomic analysis. FIG. 6A shows that treatment with UNC0638 decreases global H3K9me2 levels. A Western blot was performed of 293T whole cell extracts ±UNC0638 treatment and probed with the indicated antibodies. FIG. 6B shows a schematic diagram of the proteomic experiment. Cells grown in “light” or “heavy” media were treated with DMSO or the G9a/GLP inhibitor UNC0638 and combined prior to extracting proteins from cytoplasmic, nuclear and chromatin fractions. Cells prepared in “medium” media were treated with DMSO, processed and fractionated in parallel. Light/heavy extracts were subjected to pull-down by 3×MBT, while “medium” extracts were subjected to pull-down by 3×MBT_{D355N}. Bound proteins from each fraction were combined and analyzed by LC-MS/MS. FIG. 6C shows the identification of dozens of candidate in-cell substrates of G9a/GLP. Many proteins show decreased association with 3×MBT following 24 hour treatment of 293T cells with UNC0638 relative to DMSO treatment. Only proteins that were also enriched by 3×MBT relative to 3×MBT_{D355N} are shown. Axes show log₂ ratios for levels from vehicle control over treated cells, and each axis represents an independent biological replicate. FIG. 6D shows an MS 1 spectrum showing that the amount of WIZ, a known G9a substrate, captured by 3×MBT from cell lysate is reduced following treatment with UNC0638. The source of the relevant peak is indicated. FIG. 6E shows an MS1 spectrum showing reduction in LIG1 captured by 3×MBT following treatment with UNC0638 as in FIG. 6D.

FIGS. 7A-7D show peptide loading controls and epitope tag controls for peptide pull-downs. FIG. 7A shows dot blot of peptides used in FIG. 1B controls for peptide concentration in pull-downs. The indicated biotinylated peptides used for pull-downs in FIG. 1B were serially diluted and spotted on a nitrocellulose membrane. Peptides were detected using horseradish peroxidase-coupled streptavidin. FIG. 7B shows that N- and C-terminally Flag-tagged 3×MBT, but not methyllysine-binding mutant 3×MBT_{D355N}, binds to mono- and di-methylated H4K20-containing peptides. α-Flag Western blot analysis of peptide pull-downs with the indicated 3×MBT fusion proteins and the indicated biotinylated peptides as in FIG. 1B. Dot blot peptide loading controls are shown in FIG. 7A. The methylation binding specificity of the domain is not dependent on GST. FIG. 7C shows dot blot of peptides in FIG. 1C controls for peptide concentration in pull-downs. Blot was performed as described in FIG. 7A. FIG. 7D shows dot blot of peptides in FIG. 1D controls for peptide concentration in pull-downs. The blot was performed as described in FIG. 7A.

FIGS. 8A-8E show peptide loading controls for peptide pull-downs. FIGS. 8A-8E show peptide dot blot controls for peptide concentration in FIG. 2. The indicated peptides were serially diluted, dotted on nitrocellulose membrane, and detected using horseradish peroxidase-coupled streptavidin. Figure panels show peptides for the corresponding letter panel in FIG. 2.

FIGS. 9A-9D show that 3×MBT binds to methylated lysine in common affinity assays. FIGS. 9A and 9B show that 3×MBT, but not 3×MBT_{D355N}, recognizes endogenous histones in Far Western assays. A Far Western analysis is shown of purified calf thymus histones serially diluted to the indicated quantities as in FIG. 3B and probed with (FIG. 9A) GST-3× Flag-3×MBT and GST-3× Flag-3×MBT_{D355N} or (FIG. 9B) 3× Flag-3×MBT and 3× Flag-3×MBT_{D355N}. Binding of recombinant 3×MBT was detected with α-Flag antibody. GST is not required for 3×MBT to identify methylated histones in by Far Western analysis. *H3 indicates an H3 degradation product that migrates near H4. FIG. 9C shows in vitro methylation reactions from FIG. 2C, re-run and probed for methylation using GST-3× Flag-domain and 3× Flag-domain protein constructs in a Far Western assay. 3×MBT, but not 3×MBT_{D355N}, specifically detects monomethylated p53 by Far Western when the domain is detected through the Flag tag of the indicated recombinant domain fusion. In vitro methylation reactions were conducted with wild-type SET8 or catalytically inactive SET8_{D338A} on recombinant wild-type p53 or p53 carrying K382R substitution (p53_{K382R}). The cofactor SAM was included in the reactions as indicated. Reactions were probed with α-p53K382me1 antibody as in FIG. 2C as a positive control. Coomassie stain of p53 is shown as a loading control. FIG. 9D, in parallel to FIG. 3F, shows that 3×MBT, but not 3×MBT_{D355N}, precipitates methylated p53 from cells. Pull-down assays were performed of NE from 293T cells co-expressing SET8 or a catalytically inactive SET8_{D338A} and wild-type p53 or p53 carrying K382R substitution (p53_{K382}R) and probed with the indicated antibodies. Though SET8 overexpression appears modest, it induces a clear increase in p53K382 monomethylation.

FIGS. 10A-10E show analysis of proteins enriched by 3×MBT relative to 3×MBT_{D355N} using cellular extract divided into cytoplasmic, nuclear and chromatin fractions. FIG. 10A shows 3×MBT/3×MBT_{D355N} ratios for every protein identified in two independent experiments. Axes are log₂ and represent independent biological replicates. The 370 out of 386 proteins with average value greater than 1 are shown in dark gray. FIG. 10B shows KEGG pathways statistically enriched among the 370 proteins bound by 3×MBT in panel A. P-values are Bonferroni-corrected. FIG. 10C shows the count of proteins with selected GO cellular component terms among the 370 proteins from panel A. This indicates that candidate methylated proteins are distributed across several major cellular compartments. FIG. 10D shows a MS spectrum showing that less ACIN1, a known substrate of G9a, associates with 3×MBT following treatment with the G9a/GLP inhibitor UNC0638. ACIN1 does not appear in Table 5 because it was only identified in one biological replicate of the experiment. FIG. 10E shows that candidate physiologic G9a/GLP substrates RBM15 and MTA1 also show significant G9a-dependent signal in the ProtoArray experiment.

### DETAILED DESCRIPTION

The practice of the present invention will employ, unless otherwise indicated, conventional methods of proteomics, chemistry, biochemistry, molecular biology, and recombinant DNA techniques within the skill of the art. Such techniques are explained fully in the literature. See, e.g., A. K. Mallia, P. K. Smith, G. T. Hermanson Immobilized Affinity Ligand Techniques (Academic Press; 1^{st} edition, 1992); Affinity Chromatography: Methods and Protocols (Methods in Molecular Biology, P. Bailon ed., Humana Press; 1^{st} edition, 2000); Separation Methods In Proteomics (G. B. Smejkal and A. Lazarev eds., CRC Press; 1^{st} edition, 2005); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

### I. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “an affinity reagent” includes a mixture of two or more affinity reagents, and the like.

The term “about”, particularly in reference to a given quantity, is meant to encompass deviations of plus or minus five percent.

The terms “polypeptide,” “peptide” and “protein” are used interchangeably to refer to a polymer of amino acid residues. The terms also apply to amino acid polymers in which one or more amino acids are chemical analogues or modified derivatives of a corresponding naturally-occurring amino acids. Amino acid derivatives can include modifications to the native sequence, such as deletions, additions and substitutions (generally conservative in nature), so long as the polypeptide or peptide maintains the desired activity (e.g., binds to methylated lysine). These modifications may be deliberate, as through site-directed mutagenesis, or may be accidental, such as through mutations of hosts that produce the proteins or errors due to PCR amplification. Furthermore, modifications may be made that have one or more of the following effects: increasing affinity and/or specificity for a protein or peptide comprising a methylated lysine. Polypeptides described herein can be made recombinantly, synthetically, or in tissue culture.

A L3MBTL1 polynucleotide, nucleic acid, oligonucleotide, protein, polypeptide, or peptide refers to a molecule derived from any species. The molecule need not be physically derived from an organism, but may be synthetically or recombinantly produced. A number of L3MBTL1 nucleic acid and protein sequences are known. Representative L3MBTL1 sequences are presented in SEQ ID NO:1 and SEQ ID NO:2 and additional representative sequences are listed in the National Center for Biotechnology Information (NCBI) database. See, for example, NCBI entries: Accession Nos. NP_{—}056293, NG_{—}009238, NM_{—}015478, NM_{—}032107, XM_{—}003983531, XM_{—}004062164, XM_{—}003904731, NM_{—}001081338, NM_{—}031488, XM_{—}003253586, XM_{—}001149165, XM_{—}003936394, XM_{—}003825902, XM_{—}003787605, XM_{—}002830317, XM_{—}003756946, XM_{—}001070026, XM_{—}230849, XM_{—}002747567, XM_{—}417302, XM_{—}534423, XM_{—}002692323, XM_{—}001787381, XM_{—}003502019, XM_{—}003467762, XM_{—}002666408, and XM_{—}002721079; all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto can be used to construct a 3×MBT domain-containing affinity reagent capable of binding to a methylated protein or peptide, or a nucleic acid encoding a 3×MBT domain, as described herein.

By “fragment” is intended a molecule consisting of only a part of the intact full length sequence and structure. The fragment can include a C-terminal deletion an N-terminal deletion, and/or an internal deletion of the polypeptide. Active fragments of a particular L3MBTL1 protein or polypeptide will generally include at least one 3×MBT domain, comprising the residues corresponding to amino acids 190 to 530 of human L3MBTL1 numbered relative to the reference sequence of SEQ ID NO:2, and have the ability to bind to a methylysine (e.g., for use in an affinity reagent that binds to methylated proteins or peptides).

“Substantially purified” generally refers to isolation of a substance (compound, polynucleotide, protein, polypeptide, peptide composition) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

An affinity reagent is said to “interact” with a protein if it binds specifically (e.g., in a lock-and-key type mechanism), non-specifically or in some combination of specific and non-specific binding. An affinity reagent “interacts preferentially” with a protein if it binds to a methylated protein with greater affinity and/or greater specificity than it binds to other proteins (e.g., binds to a particular type of methylated protein (e.g., mono-methyl, di-methyl, or tri-methyl lysine) to a greater degree than to other methylated proteins or unmethylated proteins). The term “affinity” refers to the strength of binding and can be expressed quantitatively as a dissociation constant (K_{d}). In certain embodiments, the affinity reagents described herein interact preferentially with a methylated protein but, nonetheless, may be capable of binding other proteins at a weak, yet detectable, level (e.g., 10% or less of the binding shown to the protein of interest). Typically, weak binding, or background binding, is readily discernible from the preferential interaction with the compound or protein of interest, e.g., by use of appropriate controls.

The terms “label” and “detectable label” refer to a molecule capable of detection, including, but not limited to, radioactive isotopes, stable (non-radioactive) heavy isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof that is capable of exhibiting fluorescence in the detectable range. Particular examples of labels that may be used with the invention include, but are not limited to radiolabels (e.g., ³H, ¹²⁵I, ³⁵S, ¹⁴C, or ³²P), stable (non-radioactive) heavy isotopes (e.g., ¹³C or ¹⁵N), phycoerythrin, Alexa dyes, fluorescein, 7-nitrobenzo-2-oxa-1,3-diazole (NBD), YPet, CyPet, Cascade blue, allophycocyanin, Cy3, Cy5, Cy7, rhodamine, dansyl, umbelliferone, Texas red, luminol, acradimum esters, biotin or other streptavidin-binding proteins, magnetic beads, electron dense reagents, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), Dronpa, Padron, mApple, mCherry, rsCherry, rsCherryRev, firefly luciferase, Renilla luciferase, NADPH, beta-galactosidase, horseradish peroxidase, glucose oxidase, alkaline phosphatase, chloramphenical acetyl transferase, and urease. Enzyme tags are used with their cognate substrate. The terms also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), and glass microparticles with digital holographic code images (see e.g., CyVera microbeads produced by Illumina (San Diego, Calif.). As with many of the standard procedures associated with the practice of the invention, skilled artisans will be aware of additional labels that can be used.

The phrase “specifically (or selectively) binds” to an antibody or “specifically (or selectively) immunoreactive with,” when referring to a protein or peptide or epitope tag, refers to a binding reaction that is determinative of the presence of the protein in a heterogeneous population of proteins and other biologics. Thus, under designated immunoassay conditions, the specified antibodies bind to a particular protein at least two times the background and do not substantially bind in a significant amount to other proteins present in the sample. Specific binding to an antibody under such conditions may require an antibody that is selected for its specificity for a particular protein. For example, polyclonal antibodies raised to an epitope tag of an affinity reagent can be selected to obtain only those polyclonal antibodies that are specifically immunoreactive with the epitope tag of the affinity reagent and not with other proteins. A variety of immunoassay formats may be used to select antibodies specifically immunoreactive with a particular protein or epitope tag. For example, solid-phase ELISA immunoassays are routinely used to select antibodies specifically immunoreactive with a protein (see, e.g., Harlow & Lane. Antibodies, A Laboratory Manual (1988), for a description of immunoassay formats and conditions that can be used to determine specific immunoreactivity). Typically a specific or selective reaction will be at least twice background signal or noise and more typically more than 10 to 100 times background.

“Homology” refers to the percent identity between two polynucleotide or two polypeptide moieties. Two nucleic acid, or two polypeptide sequences are “substantially homologous” to each other when the sequences exhibit at least about 50% sequence identity, preferably at least about 75% sequence identity, more preferably at least about 80% 85% sequence identity, more preferably at least about 90% sequence identity, and most preferably at least about 95% 98% sequence identity over a defined length of the molecules. As used herein, substantially homologous also refers to sequences showing complete identity to the specified sequence.

In general, “identity” refers to an exact nucleotide to nucleotide or amino acid to amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M. O. Dayhoff ed., 5 Suppl. 3:353 358, National biomedical Research Foundation, Washington, D.C., which adapts the local homology algorithm of Smith and Waterman Advances in Appl. Math. 2:482 489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions.

Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non redundant, GenBank+EMBL+DDBJ+PDB+GenBank CDS translations+Swiss protein+Spupdate+PIR. Details of these programs are readily available.

Alternatively, homology can be determined by hybridization of polynucleotides under conditions which form stable duplexes between homologous regions, followed by digestion with single stranded specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

“Recombinant” as used herein to describe a nucleic acid molecule means a polynucleotide of genomic, cDNA, viral, semisynthetic, or synthetic origin which, by virtue of its origin or manipulation, is not associated with all or a portion of the polynucleotide with which it is associated in nature. The term “recombinant” as used with respect to a protein or polypeptide means a polypeptide produced by expression of a recombinant polynucleotide. In general, the gene of interest is cloned and then expressed in transformed organisms, as described further below. The host organism expresses the foreign gene to produce the protein under expression conditions.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

“Recombinant host cells”, “host cells,” “cells”, “cell lines,” “cell cultures”, and other such terms denoting microorganisms or higher eukaryotic cell lines cultured as unicellular entities refer to cells which can be, or have been, used as recipients for recombinant vector or other transferred DNA, and include the original progeny of the original cell which has been transfected.

A “coding sequence” or a sequence which “encodes” a selected polypeptide, is a nucleic acid molecule which is transcribed (in the case of DNA) and translated (in the case of mRNA) into a polypeptide in vivo when placed under the control of appropriate regulatory sequences (or “control elements”). The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

Typical “control elements,” include, but are not limited to, transcription promoters, transcription enhancer elements, transcription termination signals, polyadenylation sequences (located 3′ to the translation stop codon), sequences for optimization of initiation of translation (located 5′ to the coding sequence), and translation termination sequences.

“Operably linked” refers to an arrangement of elements wherein the components so described are configured so as to perform their usual function. Thus, a given promoter operably linked to a coding sequence is capable of effecting the expression of the coding sequence when the proper enzymes are present. The promoter need not be contiguous with the coding sequence, so long as it functions to direct the expression thereof. Thus, for example, intervening untranslated yet transcribed sequences can be present between the promoter sequence and the coding sequence and the promoter sequence can still be considered “operably linked” to the coding sequence.

“Encoded by” refers to a nucleic acid sequence which codes for a polypeptide sequence, wherein the polypeptide sequence or a portion thereof contains an amino acid sequence of at least 3 to 5 amino acids, more preferably at least 8 to 10 amino acids, and even more preferably at least 15 to 20 amino acids from a polypeptide encoded by the nucleic acid sequence.

“Expression cassette” or “expression construct” refers to an assembly which is capable of directing the expression of the sequence(s) or gene(s) of interest. An expression cassette generally includes control elements, as described above, such as a promoter which is operably linked to (so as to direct transcription of) the sequence(s) or gene(s) of interest, and often includes a polyadenylation sequence as well. Within certain embodiments of the invention, the expression cassette described herein may be contained within a plasmid construct. In addition to the components of the expression cassette, the plasmid construct may also include, one or more selectable markers, a signal which allows the plasmid construct to exist as single stranded DNA (e.g., a M13 origin of replication), at least one multiple cloning site, and a “mammalian” origin of replication (e.g., a SV40 or adenovirus origin of replication).

“Purified polynucleotide” refers to a polynucleotide of interest or fragment thereof which is essentially free, e.g., contains less than about 50%, preferably less than about 70%, and more preferably less than about at least 90%, of the protein with which the polynucleotide is naturally associated. Techniques for purifying polynucleotides of interest are well-known in the art and include, for example, disruption of the cell containing the polynucleotide with a chaotropic agent and separation of the polynucleotide(s) and proteins by ion-exchange chromatography, affinity chromatography and sedimentation according to density.

The term “transfection” is used to refer to the uptake of foreign DNA by a cell. A cell has been “transfected” when exogenous DNA has been introduced inside the cell membrane. A number of transfection techniques are generally known in the art. See, e.g., Graham et al. (1973) Virology, 52:456, Sambrook et al. (2001) Molecular Cloning, a laboratory manual, 3rd edition, Cold Spring Harbor Laboratories, New York, Davis et al. (1995) Basic Methods in Molecular Biology, 2nd edition, McGraw-Hill, and Chu et al. (1981) Gene 13:197. Such techniques can be used to introduce one or more exogenous DNA moieties into suitable host cells. The term refers to both stable and transient uptake of the genetic material, and includes uptake of peptide- or antibody-linked DNAs.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a nucleic acid of interest and which can transfer nucleic acid sequences to target cells. Thus, the term includes cloning and expression vehicles, as well as viral vectors.

The terms “variant,” “analog” and “mutein” refer to biologically active derivatives of the reference molecule that retain desired activity, such as the ability to bind to a methyl lysine of a protein or peptide. In general, the terms “variant” and “analog” refer to compounds having a native polypeptide sequence and structure with one or more amino acid additions, substitutions (generally conservative in nature) and/or deletions, relative to the native molecule, so long as the modifications do not destroy biological activity and which are “substantially homologous” to the reference molecule as defined below. In general, the amino acid sequences of such analogs will have a high degree of sequence homology to the reference sequence, e.g., amino acid sequence homology of more than 50%, generally more than 60%-70%, even more particularly 80%-85% or more, such as at least 90%-95% or more, when the two sequences are aligned. Often, the analogs will include the same number of amino acids but will include substitutions, as explained herein. The term “mutein” further includes polypeptides having one or more amino acid-like molecules including but not limited to compounds comprising only amino and/or imino molecules, polypeptides containing one or more analogs of an amino acid (including, for example, unnatural amino acids, etc.), polypeptides with substituted linkages, as well as other modifications known in the art, both naturally occurring and non-naturally occurring (e.g., synthetic), cyclized, branched molecules and the like. The term also includes molecules comprising one or more N-substituted glycine residues (a “peptoid”) and other synthetic amino acids or peptides. (See, e.g., U.S. Pat. Nos. 5,831,005; 5,877,278; and U.S. Pat. No. 5,977,301; Nguyen et al., Chem. Biol. (2000) 7:463-473; and Simon et al., Proc. Natl. Acad. Sci. USA (1992) 89:9367-9371 for descriptions of peptoids). Methods for making polypeptide analogs and muteins are known in the art and are described further below.

As explained above, analogs generally include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, and tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 conservative or non-conservative amino acid substitutions, or any integer between 5-25, so long as the desired function of the molecule remains intact. One of skill in the art may readily determine regions of the molecule of interest that can tolerate change by reference to Hopp/Woods and Kyte-Doolittle plots, well known in the art.

The term “derived from” is used herein to identify the original source of a molecule but is not meant to limit the method by which the molecule is made which can be, for example, by chemical synthesis or recombinant means.

A polynucleotide “derived from” a designated sequence refers to a polynucleotide sequence which comprises a contiguous sequence of approximately at least about 6 nucleotides, preferably at least about 8 nucleotides, more preferably at least about 10-12 nucleotides, and even more preferably at least about 15-20 nucleotides corresponding, i.e., identical or complementary to, a region of the designated nucleotide sequence. The derived polynucleotide will not necessarily be derived physically from the nucleotide sequence of interest, but may be generated in any manner, including, but not limited to, chemical synthesis, replication, reverse transcription or transcription, which is based on the information provided by the sequence of bases in the region(s) from which the polynucleotide is derived. As such, it may represent either a sense or an antisense orientation of the original polynucleotide.

### II. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention relates to methyl-lysine affinity reagents created by engineering the 3×MBT methyl-lysine binding domain of lethal (3) malignant brain tumor-like protein 1 (L3MBTL1). In particular, the invention relates to affinity reagents comprising detectable labels or tags and affinity media comprising the 3×MBT domain and methods of using such affinity reagents in detection, purification, and proteomic profiling of proteins and peptides carrying a mono- or di-methylated lysine. As the 3×MBT domain is broadly specific for methylated lysine, the affinity reagents described herein and can be universally applied to any biological system. The inventors have shown such affinity reagents can be used to detect protein methylation in proteins previously unknown to be methylated and have further shown that such affinity reagents can be used to perform proteome-wide enrichment of various methylated proteins and protein complexes (see Example 1). Their results demonstrate a powerful new approach for global and quantitative analysis of methylated lysine in biological systems.

In order to further an understanding of the invention, a more detailed discussion is provided below regarding the methyl-lysine affinity reagents and methods of using them in detection, purification, and proteomic profiling of methylated proteins and peptides.

A. Methyl-Lysine Affinity Reagents

Affinity reagents comprise one or more engineered 3×MBT domains of one or more L3MBTL1 proteins. The 3×MBT domain consists of the residues of a L3MBTL1 protein corresponding to amino acids 190 to 530 of human L3MBTL1, numbered relative to the reference sequence of SEQ ID NO:2. In one embodiment, the affinity reagent comprises at least one 3×MBT domain comprising the amino acid sequence of SEQ ID NO:29 or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto.

Nucleic acid and protein sequences that can be used to construct a methyl-lysine affinity reagent may be derived from a L3MBTL1 polynucleotide, nucleic acid, oligonucleotide, protein, polypeptide, or peptide derived from any species. The molecule need not be physically derived from an organism, but may be synthetically or recombinantly produced. A number of L3MBTL1 nucleic acid and protein sequences are known. Representative L3MBTL1 sequences are presented in SEQ ID NO:1 and SEQ ID NO:2 and additional representative sequences are listed in the National Center for Biotechnology Information (NCBI) database. See, for example, NCBI entries: Accession Nos. NG_{—}009238, NM_{—}015478, NM_{—}032107, XM_{—}003983531, XM_{—}004062164, XM_{—}003904731, NM_{—}001081338, NM_{—}031488, XM_{—}003253586, XM_{—}001149165, XM_{—}003936394, XM_{—}003825902, XM_{—}003787605, XM_{—}002830317, XM_{—}003756946, XM_{—}001070026, XM_{—}230849, XM_{—}002747567, XM_{—}417302, XM_{—}534423, XM_{—}002692323, XM_{—}001787381, XM_{—}003502019, XM_{—}003467762, XM_{—}002666408, and XM_{—}002721079; all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto can be used to construct a 3×MBT domain-containing affinity reagent capable of binding a methylated protein, or a nucleic acid encoding a 3×MBT domain, as described herein.

The 3×MBT domains and any other polypeptides included in the affinity reagent may be connected directly to each other by peptide bonds or may be separated by intervening amino acid sequences or linkers. The affinity reagent may also contain sequences exogenous to the L3MBTL1 polypeptides. For example, the affinity reagent may include targeting or localization sequences, tag sequences, or sequences of fluorescent proteins or other photochromic proteins or chromophores that can be used as a detectable label. Moreover, the affinity reagent may contain sequences from multiple L3MBTL1 proteins, or variants thereof. Alternatively, the affinity reagent may comprise only one 3×MBT domain, which can be a wild-type 3×MBT domain, or a variant thereof.

In certain embodiments, the affinity reagent comprises a linker amino acid sequence, which is typically short, e.g., 20 or fewer amino acids (i.e., 20, 19, 18, 17, 16, 15, 14, 13, 12, 11, 10, 9, 8, 7, 6, 5, 4, 3, 2, or 1). Examples include short peptide sequences which facilitate cloning, poly-glycine linkers (Glyₙ where n=2, 3, 4, 5, 6, 7, 8, 9, 10 or more), histidine tags (His. where n=3, 4, 5, 6, 7, 8, 9, 10 or more), linkers composed of glycine and serine residues ([Gly-Ser]ₙ, [Gly-Gly-Ser-Gly]ₙ (SEQ ID NO:30), [Ser-Ala-Gly-Gly]ₙ (SEQ ID NO:31), and [Gly-Gly-Gly-Gly-Ser]ₙ (SEQ ID NO:32), wherein n=1, 2, 3, 4, 5, 6, 7, 8, 9, 10, 11, 12, 13, 14, 15 or more), GSAT, SEG, and Z-EGFR linkers. Linkers may include restriction sites, which aid cloning and manipulation. Other suitable linker amino acid sequences will be apparent to those skilled in the art. (See e.g., Argos (1990) J. Mol. Biol. 211(4):943-958; Crasto et al. (2000) Protein Eng. 13:309-312; George et al. (2002) Protein Eng. 15:871-879; Arai et al. (2001) Protein Eng. 14:529-532; and the Registry of Standard Biological Parts (partsregistry.org/Protein domains/Linker).

In certain embodiments, the affinity reagent comprises an optional N-terminal amino acid sequence. This will typically be short, e.g., 40 or fewer amino acids (i.e., 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1). Examples include leader sequences to direct protein localization, or short peptide sequences or tag sequences, which facilitate cloning or purification (e.g., a histidine tag His. where n=3, 4, 5, 6, 7, 8, 9, 10 or more). Other suitable N-terminal amino acid sequences will be apparent to those skilled in the art.

In certain embodiments, the affinity reagent comprises an optional C-terminal amino acid sequence. This will typically be short, e.g., 40 or fewer amino acids (i.e., 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2, or 1). Examples include sequences to direct protein localization, short peptide sequences or tag sequences, which facilitate cloning or purification (e.g., His. where n=3, 4, 5, 6, 7, 8, 9, 10 or more), or sequences which enhance protein stability. Other suitable C-terminal amino acid sequences will be apparent to those skilled in the art.

In certain embodiments, tag sequences are located at the N-terminus or C-terminus of the affinity reagent. Exemplary tags that can be used in the practice of the invention include a glutathione S-transferase tag, a FLAG tag, a His-tag, a Strep-tag, a TAP-tag, an S-tag, an SBP-tag, an Arg-tag, a calmodulin-binding peptide tag, a cellulose-binding domain tag, a DsbA tag, a c-myc tag, a HAT-tag, a maltose-binding protein tag, a NusA tag, a biotin tag, and a thioredoxin tag.

In certain embodiments, the affinity reagent is immobilized on a solid support. Exemplary solid supports include a bead (e.g., agarose bead, polystyrene bead, microbead, magnetic bead), a particle, a rod, a membrane (e.g., a nitrocellulose membrane or a polyvinylidene difluoride (PVDF) membrane), a gel, a resin, a column, a chip, a slide, a plate (e.g., a microtiter plate), and a microarray.

In one embodiment, the invention includes an affinity chromatography matrix comprising a methyl-lysine affinity reagent bound to a solid support. Exemplary solid supports for affinity chromatography may comprise polyether sulfone, agarose, cellulose, a polysaccharide, polytetrafluoroethylene, polysulfone, polyester, polyvinylidene fluoride, polypropylene, poly (tetrafluoroethylene-co-perfluoro(alkyl vinyl ether)), polycarbonate, polyethylene, glass, polyacrylate, polyacrylamide, poly(azolactone), polystyrene, polylactide, ceramic, nylon or metal. The affinity chromatography matrix may further comprise a linking group, such as, but not limited to, cyanogen bromide, tresyl, triazine, vinyl sulfone, an aldehyde, an epoxide, or an activated carboxylic acid to facilitate coupling of the affinity reagent to the solid support. The chromatography matrix can be prepared by coupling the methyl-lysine affinity reagent to the solid support with the linking group by chemically activating the solid support if necessary, and contacting the solid support with the methyl-lysine affinity reagent such that the affinity reagent covalently attaches to the solid support. Additionally, the affinity reagent may be connected to the solid support through a linker to make the affinity reagent more accessible for binding to methylated proteins and peptides. Chromatography may be performed with the affinity chromatography matrix in a column or in batch. See, e.g., A. K. Mallia, P. K. Smith, G. T. Hermanson Immobilized Affinity Ligand Techniques (Academic Press; 1^{st} edition, 1992); Affinity Chromatography: Methods and Protocols (Methods in Molecular Biology, P. Bailon ed., Humana Press; 1^{st} edition, 2000), Guide to Protein Purification (Methods in Enzymology Vol. 182, M. P. Deutcher ed., Academic Press, Inc.); herein incorporated by reference in their entireties.

B. Production of Affinity Reagents

Affinity reagents can be produced in any number of ways, all of which are well known in the art. In one embodiment, the affinity reagents are generated using recombinant techniques. One of skill in the art can readily determine nucleotide sequences that encode the desired polypeptides to be incorporated into an affinity reagent using standard methodology and the teachings herein. Oligonucleotide probes can be devised based on the known sequences and used to probe genomic or cDNA libraries. The sequences can then be further isolated using standard techniques and, e.g., restriction enzymes employed to truncate the gene at desired portions of the full-length sequence. Similarly, sequences of interest can be isolated directly from cells and tissues containing the same, using known techniques, such as phenol extraction and the sequence further manipulated to produce the desired truncations. See, e.g., Sambrook et al., supra, for a description of techniques used to obtain and isolate DNA.

The sequences encoding polypeptides can also be produced synthetically, for example, based on the known sequences. The nucleotide sequence can be designed with the appropriate codons for the particular amino acid sequence desired. The complete sequence is generally assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311; Stemmer et al. (1995) Gene 164:49-53.

Recombinant techniques are readily used to clone sequences encoding polypeptides useful in the claimed affinity reagents that can then be mutagenized in vitro by the replacement of the appropriate base pair(s) to result in the codon for the desired amino acid. Such a change can include as little as one base pair, effecting a change in a single amino acid, or can encompass several base pair changes. Alternatively, the mutations can be effected using a mismatched primer that hybridizes to the parent nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. See, e.g., Innis et al, (1990) PCR Applications: Protocols for Functional Genomics; Zoller and Smith, Methods Enzymol. (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al. Proc. Natl. Acad. Sci USA (1982) 79:6409.

Once coding sequences have been isolated and/or synthesized, they can be cloned into any suitable vector or replicon for expression. (See, also, Example 1). As will be apparent from the teachings herein, a wide variety of vectors encoding modified polypeptides can be generated by creating expression constructs which operably link, in various combinations, polynucleotides encoding polypeptides having deletions or mutations therein.

Numerous cloning vectors are known to those of skill in the art, and the selection of an appropriate cloning vector is a matter of choice. Examples of recombinant DNA vectors for cloning and host cells which they can transform include the bacteriophage λ (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ6l (Streptomyces), pUC6 (Streptomyces), YIp5 (Saccharomyces), YCp19 (Saccharomyces) and bovine papilloma virus (mammalian cells). See, generally, DNA Cloning: Vols. I & II, supra; Sambrook et al., supra; B. Perbal, supra.

Insect cell expression systems, such as baculovirus systems, can also be used and are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit).

Plant expression systems can also be used to produce the affinity reagents described herein. Generally, such systems use virus-based vectors to transfect plant cells with heterologous genes. For a description of such systems see, e.g., Porta et al., Mol. Biotech. (1996) 5:209-221; and Hackland et al., Arch. Virol. (1994) 139:1-22.

Viral systems, such as a vaccinia based infection/transfection system, as described in Tomei et al., J. Virol. (1993) 67:4017-4026 and Selby et al., J. Gen. Virol. (1993) 74:1103-1113, will also find use with the present invention. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA that is then translated into protein by the host translational machinery. The method provides for high level, transient, cytoplasmic production of large quantities of RNA and its translation product(s).

The gene can be placed under the control of a promoter, ribosome binding site (for bacterial expression) and, optionally, an operator (collectively referred to herein as “control” elements), so that the DNA sequence encoding the desired polypeptide is transcribed into RNA in the host cell transformed by a vector containing this expression construction. The coding sequence may or may not contain a signal peptide or leader sequence. With the present invention, both the naturally occurring signal peptides or heterologous sequences can be used. Leader sequences can be removed by the host in post-translational processing. See, e.g., U.S. Pat. Nos. 4,431,739; 4,425,437; 4,338,397. Such sequences include, but are not limited to, the TPA leader, as well as the honey bee mellitin signal sequence.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector that already contains the control sequences and an appropriate restriction site.

In some cases it may be necessary to modify the coding sequence so that it may be attached to the control sequences with the appropriate orientation; i.e., to maintain the proper reading frame. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al., supra; DNA Cloning, Vols. I and II, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Vero293 cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorphs, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, polypeptides are produced by growing host cells transformed by an expression vector described above under conditions whereby the polypeptide is expressed. The selection of the appropriate growth conditions is within the skill of the art.

In one embodiment, the transformed cells secrete the polypeptide product into the surrounding media. Certain regulatory sequences can be included in the vector to enhance secretion of the protein product, for example using a tissue plasminogen activator (TPA) leader sequence, an interferon (γ or α) signal sequence or other signal peptide sequences from known secretory proteins. The secreted polypeptide product can then be isolated by various techniques described herein, for example, using standard purification techniques such as but not limited to, hydroxyapatite resins, column chromatography, ion-exchange chromatography, size-exclusion chromatography, electrophoresis, HPLC, immunoadsorbent techniques, affinity chromatography, immunoprecipitation, and the like.

Alternatively, the transformed cells are disrupted, using chemical, physical or mechanical means, which lyse the cells yet keep the recombinant polypeptides substantially intact. Intracellular proteins can also be obtained by removing components from the cell wall or membrane, e.g., by the use of detergents or organic solvents, such that leakage of the polypeptides occurs. Such methods are known to those of skill in the art and are described in, e.g., Protein Purification Applications: A Practical Approach, (Simon Roe, Ed., 2001).

For example, methods of disrupting cells for use with the present invention include but are not limited to: sonication or ultrasonication; agitation; liquid or solid extrusion; heat treatment; freeze-thaw; desiccation; explosive decompression; osmotic shock; treatment with lytic enzymes including proteases such as trypsin, neuraminidase and lysozyme; alkali treatment; and the use of detergents and solvents such as bile salts, sodium dodecylsulphate, Triton, NP40 and CHAPS. The particular technique used to disrupt the cells is largely a matter of choice and will depend on the cell type in which the polypeptide is expressed, culture conditions and any pre-treatment used.

Following disruption of the cells, cellular debris is removed, generally by centrifugation, and the intracellularly produced polypeptides are further purified, using standard purification techniques such as but not limited to, column chromatography, ion-exchange chromatography, size-exclusion chromatography, electrophoresis, HPLC, immunoadsorbent techniques, affinity chromatography, immunoprecipitation, and the like.

For example, one method for obtaining intracellular polypeptides involves affinity purification, such as by immunoaffinity chromatography using antibodies (e.g., previously generated antibodies), or by lectin affinity chromatography. Particularly preferred lectin resins are those that recognize mannose moieties such as but not limited to resins derived from Galanthus nivalis agglutinin (GNA), Lens culinaris agglutinin (LCA or lentil lectin), Pisum sativum agglutinin (PSA or pea lectin), Narcissus pseudonarcissus agglutinin (NPA) and Allium ursinum agglutinin (AUA). The choice of a suitable affinity resin is within the skill in the art. After affinity purification, the polypeptides can be further purified using conventional techniques well known in the art, such as by any of the techniques described above.

Polypeptides can be conveniently synthesized chemically, for example by any of several techniques that are known to those skilled in the peptide art (see, e.g., Fmoc Solid Phase Peptide Synthesis: A Practical Approach (W. C. Chan and Peter D. White eds., Oxford University Press, 1^{st} edition, 2000); N. Leo Benoiton, Chemistry of Peptide Synthesis (CRC Press; 1^{st} edition, 2005); Peptide Synthesis and Applications (Methods in Molecular Biology, John Howl ed., Humana Press, 1^{st} ed., 2005), herein incorporated by reference in their entireties). In general, these methods employ the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis, Synthesis, Biology, Vol. 1, for classical solution synthesis. These methods are typically used for relatively small polypeptides, i.e., up to about 50-100 amino acids in length, but are also applicable to larger polypeptides.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, o-bromobenzyloxycarbonyl, cyclohexyl, isopropyl, acetyl, o-nitrophenylsulfonyl and the like.

Typical solid supports are cross-linked polymeric supports. These can include divinylbenzene cross-linked-styrene-based polymers, for example, divinylbenzene-hydroxymethylstyrene copolymers, divinylbenzene-chloromethylstyrene copolymers and divinylbenzene-benzhydrylaminopolystyrene copolymers.

Polypeptides can also be chemically prepared by other methods such as by the method of simultaneous multiple peptide synthesis. See, e.g., Houghten Proc. Natl. Acad. Sci. USA (1985) 82:5131-5135; U.S. Pat. No. 4,631,211.

C. Applications

Methyl-lysine affinity reagents will find numerous applications in basic research and development. The affinity reagents described herein can be used to detect diverse mono- and di-methylated proteins in vitro and in vivo and will provide useful tools for proteomics research on methylation in biological systems. For example, methyl-lysine affinity reagents can be used to enrich methylated proteins from cells, for detecting and identifying methylated proteins, for monitoring lysine methyltransferase activity, and for affinity purification of methylated proteins. The affinity reagents will also find use in various methods, including, but not limited to, far-Western blotting, pull-down assays, and affinity chromatography.

For example, far-western blotting can be performed with a methyl-lysine affinity reagent to detect methylated proteins of interest. First, gel electrophoresis (e.g., SDS or native PAGE) is used to separate proteins from a sample followed by transfer of the proteins to a membrane. Methylated proteins immobilized on the membrane can then be detected by probing with a methyl-lysine affinity reagent. Optionally, blots may be treated with a cross-linking agent after probing with the methyl-lysine affinity reagent to improve detection of methylated proteins. See, e.g., Wu et al. (2007) Nat. Protoc. 2(12):3278-3284; Machida et al. (2009) Methods Mol. Biol. 536:313-329; Sato et al. (2011) J. Biosci. Bioeng. September; 112(3):304-307; herein incorporated by reference in their entireties.

In another example, pull-down assays can be performed with an affinity reagent immobilized on a solid support. The solid support may include, but is not limited to a bead (e.g., agarose bead, polystyrene bead, microbead, magnetic bead), a particle, a rod, a membrane (e.g., a nitrocellulose membrane or a polyvinylidene difluoride (PVDF) membrane), a gel, a resin, a column, a chip, a slide, a plate (e.g., a microtiter plate), and a microarray. The immobilized methyl-lysine affinity reagent is incubated with a sample containing methylated proteins (e.g., cell lysate), and the immobilized affinity reagent captures any methylated proteins or peptides present that bind to it. The solid support containing bound affinity reagent-methylated protein/peptide complexes can then be separated from the sample and any bound methylated proteins or peptides can be eluted from the affinity reagent, if desired. In this manner, pull-down assays can be used, for example, to enrich a pool of proteins in a complex mixture for methylated proteins or to isolate a methylated protein for further purification or characterization.

In another example, affinity chromatography can be performed with a methyl-lysine affinity reagent to purify a methylated protein or peptide of interest. The affinity chromatography matrix used for purification comprises an immobilized methyl-lysine affinity reagent covalently bound to the matrix, as described herein. Affinity chromatography is performed by contacting a sample (e.g., a cell lysate or partially purified mixture containing the methylated protein or peptide of interest) with the affinity chromatography matrix under a first set of conditions, such that the methylated protein or peptide binds to the immobilized affinity reagent attached to the matrix. The methylated protein or peptide is then eluted under a second set of conditions thereby purifying the methylated protein or peptide from the mixture. Affinity chromatography may be performed in a column or in batch. See, e.g., A. K. Mallia, P. K. Smith, G. T. Hermanson Immobilized Affinity Ligand Techniques (Academic Press; 1^{st} edition, 1992); Affinity Chromatography: Methods and Protocols (Methods in Molecular Biology, P. Bailon ed., Humana Press; 1^{st} edition, 2000), Guide to Protein Purification (Methods in Enzymology Vol. 182, M. P. Deutcher ed., Academic Press, Inc.); herein incorporated by reference in their entireties.

In certain embodiments, the affinity reagent may be detectably labeled to facilitate detection of the affinity reagent bound to methylated proteins or peptides. The detectable label may me any molecule capable of detection, including, but not limited to, radioactive isotopes, stable (non-radioactive) heavy isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. Particular examples of labels that may be used include, but are not limited to radiolabels (e.g., ³H, ¹²⁵I, ³⁵S, ¹⁴C, or ³²P), stable (non-radioactive) heavy isotopes (e.g., 13C or 15N), phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin or other streptavidin-binding proteins, magnetic beads, electron dense reagents, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), Dronpa, Padron, mApple, mCherry, rsCherry, rsCherryRev, firefly luciferase, Renilla luciferase, NADPH, beta-galactosidase, horseradish peroxidase, glucose oxidase, alkaline phosphatase, chloramphenical acetyl transferase, and urease. Enzyme tags are used with their cognate substrate. Detectable labels may also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), and glass microparticles with digital holographic code images (see e.g., CyVera microbeads produced by Illumina (San Diego, Calif.). Skilled artisans will be aware of additional labels that can be used.

D. Kits

Methyl-lysine affinity reagents can be provided in kits with suitable instructions and other necessary reagents for preparing or using them, as described above. The kit may contain in separate containers an affinity reagent, or recombinant constructs for producing an affinity reagent, and/or cells (either already transfected or separate). Additionally, instructions (e.g., written, tape, VCR, CD-ROM, DVD, etc.) for preparing or using the affinity reagents may be included in the kit. The kit may also contain other packaged reagents and materials (e.g., transfection reagents, buffers, media, solid support, column, and the like). Additionally, kits may comprise reagents for performing a far Western, a pull-down assay, or affinity chromatography.

In one embodiment, the kit comprises an affinity chromatography matrix comprising an affinity reagent or reagents for preparing such an affinity chromatography matrix (e.g., affinity chromatography resin, activating agent), as described herein. Kits may further comprise reagents for performing affinity chromatography (e.g., column, solutions for binding or elution of a methylated protein or peptide).

### III. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

### A General Molecular Affinity Tool and Strategy for Global Detection and Proteomic Analysis of Lysine Methylation

Here we describe an affinity reagent engineered from the three Malignant Brain Tumor domain repeats (3×MBT) of L3MBTL1 and show that it can serve as a tool for detecting, enriching, and identifying mono- and di-methylated lysine on individual proteins and on a proteomic scale. This reagent is highly specific for mono- and di-methylated lysine, and it binds to these residues with essentially no dependence on the surrounding protein sequence (Li et al. (2007) Mol. Cell 28, 677-691; Min et al. (2007) Nat. Struct. Mol. Biol. 14, 1229-1230; Nady et al. (2012) J. Mol. Biol. 423, 702-718). The 3×MBT domain can be used as a global affinity reagent to detect methylated lysine on a wide range of protein and peptide targets. We have used this approach to show that the lysine deacetylase SIRT1 is methylated in vivo by G9a (also called EHMT2 and KMT1C), and that 3×MBT can be used for screening and identifying potential G9a methylated substrates on a large-scale protein array platform (Levy et al. (2011) Epigenetics Chromatin 4, 19). In proteome-wide pull-down 3×MBT specifically enriches over three hundred proteins and allows direct identification of methylated lysine on a subset of those proteins. Finally, we have developed a cell-based proteomic strategy for substrate discovery of lysine methylation regulatory enzymes. We have used this strategy to identify over twenty known and candidate substrates of the KMTs G9a and GLP (Rathert et al. (2008) Nat. Chem. Biol. 4, 344-346) in cells by examining changes in global lysine methylation following treatment with a specific inhibitor of these enzymes (Kubicek et al. (2007) Mol. Cell 25, 473-481); Vedadi et al. (2011) Nat. Chem. Biol. 7, 566-574). This is the first proteome-wide analysis of methylated lysine. Our approach will provide a powerful new tool for studying the systems biology of lysine methylation.

Experimental Procedures

Materials

The three MBT repeats of L3MBTL1 (amino acids 190-530 of accession NP_{—}056293.4) were expressed as a GST fusion from the pGEX6P1 vector (West et al. (2010) J. Biol. Chem. 285, 37725-37732; herein incorporated by reference). Recombinant proteins were expressed and purified as previously described (West et al., supra).

MBT Far Western and Protein Pull-Down

For Far Western assays, proteins were separated by SDS-PAGE and transferred to a PVDF membrane. Following blocking, membranes were incubated overnight with the indicated domain. Domain binding was determined using an epitope tag-specific antibody and appropriate HRP-conjugated secondary antibody.

GSH-Sepharose coupled domain beads were generated for pull-down by incubating GSHSepharose resin with E. coli lysate containing sufficient 3×MBT for bead saturation. Bead-coupled 3×MBT was incubated with nuclear extract overnight and proteins that precipitated with the resin were analyzed by SDS-PAGE.

SIRT1 Screen and Characterization of Methylation

Recombinant GST-SIRT1 was expressed by baculoviral transduction of the Sf9 insect cell line as previously described (Levy et al. (2011) Nat. Immunol. 12, 29-36). A panel of enzymes was tested for ability to methylate recombinant SIRT1 using ³H-SAM in in vitro methylation reactions (Kuo et al. (2011) Mol. Cell 44, 609-620). SIRT1_{K622R} was generated by site-directed mutagenesis (Stratagene). For the pull-down experiment, 293T cells were transfected with a 10:1 enzyme:substrate DNA ratio. 150 μg of nuclear proteins were used for pull-downs with 3×MBT or 3×MBT_{D355N} coupled GSH-Sepharose as described above.

Protein Array

The assay was conducted as previously described (Levy et al. (2011) Epigenetics Chromatin 4, 19). Briefly, a ProtoArray (Invitrogen, version 5.0) was incubated overnight with 50 μg of GST or recombinant GSTtagged G9a SET domain and the cofactor SAM. Methylation was visualized by probing with 3× Flag-3×MBT, followed by α-Flag M2 antibody and an α-mouse fluorescent secondary antibody.

Protein Pull-Down and Mass Spectrometry

Protein pull-downs were performed as described above using 600 μg of nuclear extract prepared with isotopic labels as indicated. After eluting with glutathione, 3×MBT domain was removed by dialysis into high salt and rebinding to GSH-sepharose. Bound proteins were separated by SDS-PAGE and analyzed by in-gel tryptic digest followed by LC-MS/MS. Peptides were identified using MaxQuant version 1.2.2.5 (Cox and Mann (2008) Nat. Biotechnol. 26, 1367-1372) and candidate methylated peptides were verified by manual inspection.

Supplemental Experimental Procedures

Plasmid Vectors, Cell Culture and Transfections

In GST-domain-Flag constructs, a Flag epitope sequence was inserted immediately following amino acid 530 before translation termination. In GST-3× Flag-domain constructs, the 3× Flag sequence was inserted immediately following the PreScission Protease recognition sequence. 3× Flag-domain and domain-Flag constructs were generated by PreScission Protease cleavage of the GST-containing versions.

Mammalian expression vectors for SET8, p53 SETD6 and RelA were as described (Levy et al., supra; Shi et al., supra). pcDNA3.1 and p3×FLAG-CMV-7 were used for mammalian expression of G9a and SIRT1 respectively.

HEK 293T cells were grown in Dulbecco's Modified Eagle Medium (DMEM) supplemented with 10% new-born calf serum (Atlanta Biologicals), L-glutamine (Gibco), non-essential amino acids (Gibco), and penicillin/streptomycin (Gibco). Expression vectors were transfected into HEK 293T cells using TransIT-293 transfection reagent (Mirus) according to the manufacturers protocol. Nuclear protein extracts were prepared by hypotonic lysis and high-salt extraction (Sigma-Aldrich). Where applicable chromatin-bound proteins were extracted following nuclear protein extraction by treating pellets with 1 unit of micrococcal nuclease (Sigma-Aldrich) in 50 mM Tris pH 8.0 with 5 mM CaCl₂ for 30 minutes at room temperature.

Antibodies

α-GST: Covance, α-GST(HRP): Abcam ab3416, α-GST for peptide array: Abcam ab19256, fluorescent secondary for peptide array: Invitrogen Alexa Fluor 647 A21443, fluorescent secondary for ProtoArray: Invitrogen Alexa Fluor 555 A21424, α-p53K382me1 as described (Shi et al., supra), α-H3K9me2: Abcam ab1220, α-H3: Covance, α-p53: Calbiochem OP43, α-RelAK310me1 and α-SETD6 as described (Levy et al., supra), α-RelA: Abcam ab16502, α-Flag M2: Sigma F1804, streptavidin-HRP: Jackson ImmunoResearch Laboratories, Inc. 016-030-084, α-SET8: Abcam ab3744, α-H4K9me2: Cell Signaling Technology 4658.

Peptide Pull-Down and Peptide Array Assays

Peptide pull-down assays were performed as previously described (Shi et al. (2006) Nature 442, 96-99), using 1 μg protein and 1 μg peptide per condition in binding buffer (50 mM Tris pH 7.5, 150 mM NaCl, 0.05% NP-40). Peptide arrays were performed as previously described (Bua et al., supra).

MBT Far Western and Protein Pull-Down

For Far Western assays on purified HeLa nucleosomes and calf thymus histones, the indicated amounts of nucleosome or histone were separated by SDS-PAGE and transferred to a PVDF membrane. The membrane was blocked in milk prepared with Tris-buffered saline with 0.1% Tween (TBST). Membranes were incubated overnight at 4° C. with 1 μg/ml of the indicated domain in TBST. Domain binding was determined using an epitope tag-specific antibody and appropriate HRP-conjugated secondary antibody.

For the SET8/p53 Far Westerns, p53 was methylated in vitro as previously described (Kuo et al., 2011), with 5 μg enzyme and 1 μg substrate per 25 μl of reaction volume incubated at 30° C. overnight. Following the methylation reaction, PreScission Protease was added directly to the reaction mix and samples were incubated overnight at 4° C. Blotting was conducted as described above. G9a SET domain/recombinant H3 (New England BioLabs) samples were prepared similarly, without a GST cleavage step.

GSH-Sepharose coupled domain beads were prepared for pull-down by expressing the domain in E. coli. Cells were lysed as described, and lysate was incubated overnight at 4° C. with GSH-Sepharose with a sufficient lysate-to-Sepharose ratio to result in Sepharose binding saturation. The GSH-Sepharose was then washed 3×5 minutes in binding buffer and resuspended in binding buffer to 50% slurry. 150 μg of nuclear proteins were incubated overnight at 4° C. with 15 μl 50% slurry of either 3×MBT or 3×MBT_{D355N} coupled to GSH-Sepharose in binding buffer (total volume of about 150 μl). Beads were washed 3×5 minutes in 0.5 ml of binding buffer. Beads were boiled in 25 μl of 2× Laemmli buffer and supernatant was analyzed by SDS-PAGE and Western blotting.

For SETD6/RelA and SET8/p53 pull-down experiments, 293T cells were transfected with a 1:1 enzyme:substrate DNA ratio. 150 μg of nuclear proteins were incubated overnight at 4° C. with 15 μl 50% slurry of either 3×MBT or 3×MBT_{D355N} coupled to GSH-Sepharose in binding buffer. Beads were washed 3×5 minutes in 0.5 ml of binding buffer. Proteins were subsequently eluted by adding 20 μl of PreScission Protease cleavage buffer (50 mM Tris pH 7.0, 150 mM NaCl, 1 mM EDTA, 1 mM DTT) with the protease and incubating overnight at 4° C. Laemmli buffer was added to the supernatant to 1×, and the resulting samples analyzed by SDS-PAGE and Western blotting.

Protein Array Analysis

Arrays were analyzed by scanning wavelengths 532 nm (3× Flag-3×MBT, α-Flag M2, α-mouse Alexa Fluor 555) and 635 nm (array orientation features) using an Axon GenePix 4000B microarray scanner (Molecular Devices) at 10 μm resolution. Arrays were analyzed using GenePix Pro 6.1 software (Molecular Devices) and feature values were assigned to proteins using the array list provided by Invitrogen. We designated the following criteria for “hits”: GST array signal-to-noise ratio (SNR) less than or equal to 1.5, G9a array SNR greater than or equal to 10, and a difference between the G9a array SNR and the GST array SNR of greater than or equal to 12. Hits were individually visually validated. Proteins were removed from the list of potential substrates if irregular array features appeared to interfere with the array spots or SNR calculations.

Proteomic Pull-Down and in-Gel Digestion

For stable isotope labeling by amino acids in cell culture (SILAC) experiments HEK 293T cells were grown for at least six doublings in DMEM for SILAC supplemented with 10% dialyzed fetal bovine serum (FBS), 230 mg/L L-proline and either 100 mg/L each of L-lysine and L-arginine (“light” medium), D₄-L-lysine and ¹³C₆-L-arginine (“medium” media), or ¹³C₆¹⁵N₂-L-lysine and ¹³C₆¹⁵N₄-L-arginine (Thermo Scientific). Heavy methyl labeling used DMEM without L-Methionine or L-Cysteine supplemented with D₃¹³C-L-Methionine (Sigma), L-Cysteine (Sigma) and 10% dialyzed FBS.

600 μg of nuclear extract was incubated overnight rotating at 4° C. with 20 μl packed 3×MBT or 3×MBT_{D355N} beads in a total volume of 250 μL with binding buffer. Supernatant was removed and saved for use as a loading control. Beads were washed 4×5 minutes with 0.5 mL binding buffer. Protein was eluted by incubating in 20 μL elution buffer (10 mg/mL glutathione in 50 mM Tris pH 8.0) rotating at 4° C. for four hours. Elutions from 3×MBT and 3×MBT_{D355N} were combined and dialyzed overnight at 4° C. against 10,000× volume of dissociation buffer (50 mM Tris pH 7.5, 750 mM NaCl) in a 0.1 mL Slide-A-Lyzer MINI Dialysis Device with 3.5K molecular weight cutoff (Thermo Scientific). 80 μL of GSH-Sepharose was prepared by washing briefly three times with 0.5 mL binding buffer. Dialyzed proteins were added to the GSH-Sepharose and incubated 4 hours rotating at 4° C. to deplete the GST-fusion domains. Supernatant was transferred to a fresh 1.5 mL tube. Proteins were separated by SDS-PAGE on a 10% polyacrylamide gel and stained using SilverQuest Silver Staining Kit (Life Technologies). Lanes were excised, residual 3×MBT band was removed, and gel was diced into 0.5 mm cubes. Gel pieces were destained and processed by in-gel digestion (Carlson et al. (2011) Science Signaling 4) with sequencing grade trypsin (Promega) and the peptide sample was dried completely in a vacuum centrifuge. To prepare a loading control, 2 μL each of light and heavy nuclear extract were combined, 6 μL of 8M urea was added, proteins were denatured by adding 2 μL of 100 mM dithiothreitol in digest buffer (50 mM ammonium bicarbonate) and heating to 60° C. for 1 hour, cysteine was alkylated by adding 2.2 μL 100 mg/mL iodoacetamide in digest buffer and incubating 1 hour at room temperature in darkness, and proteins were digested by adding 80 μL trypsin (Promega) at 12.5 ng/μL in digest buffer and incubating overnight at room temperature in darkness. Digestion was stopped by adding 10 μL of 10% formic acid.

Proteomic Experimental Design

Each experiment consisted of two sets of pull-downs: “forward” labeling with 3×MBT incubated with light nuclear extract and 3×MBT_{D355N} with medium extract, and a second “reverse” labeling experiment with the label conditions swapped. Proteins were included in the results if they were quantified in both labeling directions. To determine reproducibility the entire experiment including the label swap was duplicated using independent protein preparations and a mass spectrometer at another institution.

For UNC0638 treatments cells were treated for 24 hours with UNC0638 at 2.5 μM or DMSO vehicle control. One experiment treated cells in light media with UNC0638, the other treated cells in heavy media. Both experiments included cells in medium media treated with DMSO for negative control pull-down with 3×MBT_{D355N}.

Mass Spectrometry

For 3×MBT pull-down experiment #1 the gel lane was processed and peptides were loaded off-line onto a first dimension trap column (Waters Xbridge, C18, 10 μm particle size, 100 Å pore size, 5 cm packing length, 150 μm column i.d.). Online peptide separation coupled to MS/MS used a 2D-nanoLC system (nanoAcquity UPLC system, Waters Corp., Milford, Mass., USA) equipped with a third, 6-port, 2-position valve (VICI Valco, Houston, Tex.) and Velos-Pro/Orbitrap-Elite hybrid mass spectrometer (ThermoFisher Scientific). Six discrete elutions were performed at 0.5 μL/min with 5 mM ammonium formate pH 10 using increasing concentrations of acetonitrile (5%, 10%, 15%, 20%, 25%, 30% and 42% V/V) and diluted with 9.5 ml/min 0.1% formic acid, pH 3, prior to loading onto a secondary dimension trap column (2DT) (YMC A, C18 10 μm particle size, 100 Å pore size, 10 cm packing length, 100 μm column i.d.) connected to an analytical column with an incorporated electrospray emitter (YMC-AQ, C18, 5 μm particle size, 100 Å pore size, 20 cm packing length, 50 μm column i.d., 1 μm tip diameter. Peptide separation was achieved using a gradient from 3 to 80% (V/V) of acetonitrile in 0.1% formic acid over 115 minutes with nanoflow at −20 μl/minute achieved with a passive split prior to the second dimension.

The mass spectrometer was operated in data-dependent mode using a Top 10 method. Full MS scans (m/z 300-2000) used the Orbitrap analyzer (resolution=120,000). MS/MS scans in the ion trap analyzer used collision induced dissociation with normalized collision energy of 35% and multistage activation to account for neutral loss of water. Ion selection threshold was 500 counts for charge state precursors 2 to 5 and isolation window was 2.

Experiment #2 was conducted at the Stanford University Vincent Coates Foundation Mass Spectrometry Laboratory. The gel lane was divided above and below 75 kD and analyzed separately. Nano reversed phase HPLC used a Proxeon easy nanoLC (Thermo Fisher Scientific) with buffer A consisting of 0.585% acetic acid in water and buffer B 0.585% acetic acid in 98% acetonitrile. New Objective fused silica column self-packed with duragel C18 (Peeke, Redwood City, Calif.) matrix was used with a linear gradient from 2% B to 40% B over two hours at a flow rate of 300 nL/minute. An LTQ Orbitrap Velos (Thermo Fischer Scientific) was set in data dependent acquisition mode to perform MS/MS on the top twelve most intense multiply-charged ions.

UNC0638 treatment experiments used a Q-Exactive mass spectrometer (ThermoFisher Scientific). A 25 cm HPLC column with 3 μm Reliasil (Orochem) was used to separate peptides over a 4 hour gradient from 0 to 75% acetonitrile. Ions were generated using a custom nanospray source flowing at 250 nL/minute. Mass spectrometry settings were 70,000 MS1 resolution, data dependent selection of up to 20 precursor ions per cycle, dynamic exclusion of 30 seconds and isolation window of 2 m/z. The nuclear fraction from the experiment using UNC0638 on cells in heavy media was additionally analyzed by 2D HPLC-MS/MS as described earlier.

Mass Spectrometry Data Processing

Peptides were identified and quantified using MaxQuant version 1.2.2.5 (Cox and Mann, supra) with the following changes to default settings: mono- and di-methylation of lysine, and oxidation of methionine, and N-terminal acetylation as variable modifications, disabled “I=L”, up to three modifications per peptide, one missed cleavage, maximum charge of 5. Protein and peptide FDR were set to 1%, and proteins were filtered post-hoc for at least two unique peptides. Proteins were excluded from an experiment if they were not quantified in both labeling directions. Because FDR is unreliable for small numbers of peptides the modification site FDR was set to 10% and all methylated peptides were manually verified. Methylated peptides identified from mixed spectra, or in which a similar non-methylated peptide would have indistinguishable mass, were excluded. Quantitative protein data was normalized to the mean value among proteins identified by analyzing 50 ng of digested loading control.

For UNC0638 experiments the treated vs. untreated ratio was determined by subtracting the value from 3×MBT_{D355N} prior to dividing values from the light and heavy channels. Only proteins showing a two-fold signal to background ratio (3×MBT/3×MBT_{D355N}) were evaluated as potential G9a/GLP substrates.

Bioinformatics

A threshold of 2 was used to select proteins for functional analysis because it corresponds to 313 proteins enriched by 3×MBT compared to 3 proteins enriched at the same threshold by 3×MBT_{D355N}, corresponding to a false-discovery rate of approximately 1%.

The DAVID Bioinformatics Resource version 6.7 was used to identify GO Terms enriched among proteins enriched by 3×MBT, using proteins identified in input material as background (Dennis et al. (2003) Genome Biol 4, P3; Huang et al. (2009) Nat. Protoc. 4, 44-57; herein incorporated by reference). Protein interaction network was generated using the STRING database version 9.0 (Szklarczyk et al., supra). Interactions were filtered for highest confidence (>0.900) using Experiments, Databases and Text Mining.

Results

3×MBT Recognizes Methylated Lysine with Broad Sequence Specificity

We first aimed to identify a protein domain with broad specificity for methylated lysine. Based on available structural and biochemical data (Li et al., supra; Min et al., supra; Nady et al., supra) we postulated that the 3×MBT domain of L3MBTL1 would be likely to act as a pan-specific reagent for mono- and di-methylated lysine. In its biological context L3MBTL1 plays a role in chromatin compaction mediated by interactions with histone 4 mono- and di-methylated at lysine 20 (H4K20me1/2) and histone H1B mono- and di-methylated at lysine 26 (H1BK26me1/2) (Trojer et al. (2007) Cell 129, 915-928). It also binds to methylated non-histone proteins such as p53, pRb, and the DNA replication machinery (Gurvich et al. (2010) Proc. Natl. Acad. Sci. U.S.A. 107, 22552-22557; Saddic et al. (2010) J. Biol. Chem. 285, 37733-37740; Trojer et al., supra; West et al. (2010) J. Biol. Chem. 285, 37725-37732). Previous work has shown that the domain interacts with methylated lysine through a hydrophobic binding pocket, and that hydrogen bonding between the carboxylate of aspartate 355 and the methylammonium proton of mono- or di-methyl lysine confers specificity over tri-methylation (Li et al., supra; Min et al., supra). Mutation of aspartate 355 to asparagine (3×MBT_{D355N}) has been shown to abrogate binding to methyl-lysine without affecting the overall structure of the domain (Li et al., supra; Min et al., supra). Importantly, the isolated 3×MBT domain forms no significant contacts with the side-chains of amino acids surrounding the methylated residue, providing a structural basis for sequence-independent binding to methyl-lysine. The specificity of L3MBTL1 for physiologically-relevant methylated proteins such as H4 and Rb is thought to be conferred by the 3×MBT binding to methyl-lysine in combination with non-methyl sensitive interactions mediated by regions outside of the 3×MBT domain (Trojer et al., supra).

We first expressed the isolated 3×MBT domain of L3MBTL1 and the 3×MBT_{D355N} mutant as GST fusions (FIG. 1A) and characterized their binding to methylated peptides. As in previous studies, the wild-type domain, but not the point-mutant, co-precipitates with mono- and di-methylated peptides from p53 and H4, both known binding partners of L3MBTL1 (FIG. 1B; FIG. 7A) (Min et al., supra; Nady et al., supra; West et al., supra). This interaction is largely independent of GST (e.g. Flag can be used as the epitope tag as well) and can be detected through both N- and C-terminal tagging of the domain (FIG. 7B). 3×MBT also co-precipitates with thirteen different mono- and di-methylated peptides drawn from variety of histone and nonhistone proteins (FIG. 1C; FIG. 7C). In contrast, 3×MBT does not bind a series of non-methylated peptides, and binds very weakly to some tri-methylated peptides (FIG. 1D; FIG. 7D). We next probed 3×MBT on a peptide array displaying over 100 unique peptides with different post-translational modifications (PTMs) (Bua et al. (2009) PLoS One 4, e6789; Kuo et al. (2012) Nature 484, 115-119). The domain bound specifically to the 41 peptides containing mono- and di-methylated lysine, and did not bind to the dozens of unmodified peptides, peptides containing tri-methylated lysine, or peptides with other PTMs (FIG. 1E). Together, these data demonstrate that highly preferential binding of 3×MBT to mono- and di-methylated lysine is a generalizable characteristic across diverse amino acid sequences.

Secondary modification of adjacent residues are well known to affect the binding affinity of many methyl-lysine binding proteins and antibodies that recognize specific methylated lysines (Bock et al. (2011) BMC Biochem 12, 48; Dhayalan et al. (2011) Chem Biol 18, 111-120; Fuchs et al. (2011) Curr. Biol. 21, 53-58; Levy et al. (2011) Nat. Immunol. 12, 29-36; Ramon-Maiques et al. (2007) Proc. Natl. Acad. Sci. U.S.A. 104, 18993-18998; Rothbart et al. (2012) Nat. Struct. Mol. Biol. 19, 1155-1160; Varier et al. (2010) EMBO J 29, 3967-3978). To test whether nearby lysine acetylation, serine phosphorylation, and arginine methylation events influence binding of 3×MBT to mono- and di-methylated lysine, peptide pull-down assays were performed with 3×MBT and a series of singly- or doubly-modified peptides (FIGS. 2A-2E; see FIGS. 8A-8E for peptide loading controls). Consistent with previous reports (Li et al. (2007) Mol. Cell 28, 677-691), phosphorylation on a C-terminally adjacent serine decreased, but did not eliminate, binding of 3×MBT to mono- or di-methylated lysine (FIGS. 2A and 2B). Arginine methylation one or two residues removed from the lysine methylation site had little effect on binding by 3×MBT (FIGS. 2C and 2D). Similarly, acetylation of a neighboring lysine and phosphorylation of an N-terminally adjacent serine did not affect 3×MBT binding (FIG. 2E). We conclude that 3×MBT preferentially recognizes mono- or di-methylated lysine even in the presence of several different types of common secondary post-translational modifications.

3×MBT Recognizes Methylated Proteins by Far Western Analysis and in Pull-Down Assays

Two common uses of modification-specific antibodies are Western blotting and immunoprecipitation (IP). We therefore asked whether the 3×MBT domain could be adapted in lieu of an antibody for these types of techniques. In Far Western assays 3×MBT, but not 3×MBT_{D355N}, detected histones from purified HeLa nucleosomes (FIG. 3A) and calf thymus histones (FIG. 3B), suggesting that the intact methyl-lysine binding pocket specifically recognizes endogenous methylated histones.

To test if the Far Western signal is indeed specific for methylated lysine, we probed for in vitro methylation of recombinant proteins (FIGS. 3C and 9C). 3×MBT, but not 3×MBT_{D355N} detected recombinant p53 when mono-methylated at lysine 382 by SET8 (also named PR-Set7 and SETD8), similar to a previously described anti-p53K382me1 antibody (Shi et al. (2007) Mol. Cell 27, 636-646). This binding is lost when the methylation event is blocked by mutating p53 lysine 382 to arginine, by using a catalytically-inactive SET8 mutant, or by withholding the methyl-donating cofactor S-adenosyl methionine (SAM) (FIGS. 3C and 9C). Similarly, 3×MBT recognizes recombinant H3 in a Far Western when H3 is dimethylated by G9a at lysine 9 (FIG. 3D) (Shinkai and Tachibana (2011) Genes Dev. 25, 781-788). Based on these data we conclude that the 3×MBT can be used in Far Western assays to detect various mono- and di-methylation events.

We next asked whether 3×MBT could specifically capture methylated proteins from cellular extract. Immobilized 3×MBT precipitates both H3 (an abundant methylated protein) and p53 (a low abundance methylated protein) from nuclear extracts (FIG. 3E). To test if the pull-down by 3×MBT is methylation-dependent, pull-downs were conducted using nuclear extract from 293T cells co-transfected with the KMT SETD6 and its substrate RelA; SETD6 mono-methylates RelA at lysine 310 (RelAK310me1) (Levy et al. (2011) Nat. Immunol. 12, 29-36). RelA was precipitated by 3×MBT in a manner dependent upon intact K310 and expression of wild-type SETD6 (FIG. 3F). Similar results were observed in 3×MBT pull-downs of methylated p53 from cells overexpressing p53 and SET8 (FIG. 9D). Taken together, we conclude that 3×MBT can be used to affinity purify methylated proteins from cellular extracts.

Utility of 3×MBT to Identifj Previously Uncharacterized Methylation Events

As the generation of site- and state-specific antibodies for the analysis of candidate novel lysine methylation events is expensive and often challenging (Egelhofer et al. (2011) Nat. Struct. Mol. Biol. 18, 91-93; Fuchs et al. (2011) Curr. Biol. 21, 53-58), we reasoned that the 3×MBT domain could serve as a readily available and inexpensive reagent to initiate investigation of newly identified methylated proteins. Through in vitro screening of a panel of enzymes as described in (Levy et al. (2011) Nat. Immunol. 12, 29-36), we found that the KMT G9a methylates the NAD-dependent lysine deactylase SIRT1 at lysine 622, a previously uncharacterized modification event (FIGS. 4A and 4B; data not shown). We next asked whether G9a catalyzes this reaction in vivo. We prepared extracts from 293T cells expressing G9a and 3× Flag-SIRT1 or 3× Flag-SIRT1_{K622R} for pull-down assays using 3×MBT and 3×MBT_{D355N} affinity resins. We found that 3× Flag-SIRT1 is specifically precipitated by 3×MBT, but not 3×MBT_{D355N}, only when G9a is co-expressed and the SIRT1 target lysine K622 is intact (FIG. 4C). These data indicate that SIRT1 is methylated by G9a in cells and demonstrate how 3×MBT can be used to uncover and characterize new biological methylation events without requiring the time- and cost-consuming generation of a methyl-specific antibody.

We next asked whether 3×MBT could be used to identify other potential G9a substrates in the context of an on-chip protein array methylation system that we previously described (Levy et al. (2011) Epigenetics Chromatin 4, 19). On-chip in vitro methylation assays using recombinant GST-G9a SET domain, or as a negative control, GST alone, were performed on Invitrogen ProtoArrays bearing 9,500 unique recombinant human proteins (FIG. 4D). Positively methylated proteins were detected by probing the arrays with Flag-tagged 3×MBT, followed by α-Flag antibody and fluorescent secondary antibody. Using a very strict threshold (see Supplemental Experimental Procedures) followed by manual inspection, we identified 112 proteins that were detected by Flag-3×MBT on the G9a-methyated array but not on the control array, indicating that these hits are potential G9a substrates (FIGS. 4D and 4E; Table 3). Previously, G9a targets could not be identified in the protein array format using antibodies because of restricted affinity of antibodies recognizing di-methylated lysine, the state catalyzed by G9a. Thus, our results demonstrate that 3×MBT can be used in combination with a protein array platform to elucidate candidate substrates of mono- and di-methyltransferase KMTs.

We note that several previously reported G9a targets were not detected in the ProtoArray® experiment, likely due to a combination of limitations associated with protein array approaches, including (1) absence of the target, (2) presentation of substrates in vitro on arrays versus in cells, and (3) other inherent issues with protein arrays such as candidate substrates being truncated proteins or improperly folded (Levy et al. (2011) Epigenetics Chromatin 4, 19). Thus, in vitro protein array approaches are likely to be most effective in helping focus on substrates when combined with other strategies, such as chemical biological approaches (Islam et al. (2012) J. Am. Chem. Soc. 134, 5909-5915; Islam et al. (2011) ACS Chem. Biol. 6, 679-684), and cell-based assays for KMT substrate identification such as the proteomic strategy described below.

3×MBT Enriches the Methyl-Lysine Proteome

We postulated that we could apply quantitative mass spectrometry in combination with the differential methyl-lysine recognition properties of 3×MBT versus the 3×MBT_{D355N} mutant to enrich an entire lysine methylation proteome (see schematic, FIG. 5A). To this end, we first performed protein pull-downs from nucleoplasmic extract of 293T cells using the 3×MBT domain or the 3×MBT_{D355N} negative control. In multiple independent experiments, 3×MBT consistently captured more proteins than 3×MBT_{D355N} (FIG. 5B), indicating that the intact methyl-lysine binding pocket is responsible for a large fraction of bound proteins.

We next used stable isotopic labeling by amino acids in cell culture (SILAC) (Ong et al. (2002) Mol. Cell Proteomics 1, 376-386) and liquid chromatography coupled to tandem mass spectrometry (LC-MS/MS) to quantitatively measure proteins enriched by binding to 3×MBT relative to the 3×MBT_{D355N} negative control (see schematic, FIG. 5A). This experiment consisted of protein pull-downs in which immobilized 3×MBT or 3×MBT_{D355N} was incubated with proteins containing either L-lysine/L-arginine or D4-L-lysine/¹³C6-L-arginine (“light” and “heavy” respectively), and proteins bound in each pull-down were combined for analysis by LC-MS/MS. We conducted “forward” and “reverse” labeling experiments where 3×MBT was matched with heavy or light lysate respectively, and only proteins observed in both labeling directions were included in the dataset for downstream analysis. The entire label swap experiment was conducted twice to determine inter-experiment reproducibility (total of four mass spectrometry analyses). A total of 544 proteins were identified in at least one pair of label-swap experiments. The SILAC ratio for 3×MBT pull-down relative to 3×MBT_{D355N} was highly correlated between the two experiments (R²=0.86, FIG. 5C), showing that enrichment is quantitative and reproducible.

We found that a large fraction of proteins were quantitatively enriched by 3×MBT relative to 3×MBT_{D355N}. For example, 313 of the 544 proteins (57.5%) were enriched at 2-fold by 3×MBT versus the mutant while only 3 of 544 proteins (0.6%) exceeded that threshold in the other direction (FIG. 5D). For comparison, 0.4% of proteins identified in the input material fell outside a SILAC ratio of 2. These data strongly argue that the proteins identified in the 3×MBT pull-down are enriched in a methyl-dependent manner.

For functional analysis of enriched proteins we used the set of 313 proteins enriched 2-fold by 3×MBT. Gene Ontology (GO) and the String protein interaction database were used to identify biological functions and protein complexes over-represented among enriched proteins relative to their frequency among proteins identified in the input material (see Methods). Overrepresented GO terms include mRNA processing (enriched at 2.9-fold, p=7e-20), transcription (2.1 fold, p=1.5e-4), and RNA and DNA helicase activity (enriched at 4.3-fold and 4.0-fold, p=0.014 and 0.011) (all p-values Bonferroni-corrected, Table 4) (Huang et al., 2009a, b). Analysis of protein interactions using the String Database shows that proteins bound by the 3×MBT domain group into related clusters (FIG. 5E) (Szklarczyk et al. (2011) Nucleic Acids Res. 39, D561-568), the largest of which is composed of proteins involved in RNA processing. Smaller clusters include the DNA replication-associated minichromosome maintenance helicase complex, proteins involved in the DNA damage response, and a number of chromatin modifying complexes.

Quantitative Analysis of the Dynamic Methyl-Lysine Proteome

A powerful application of proteome-wide analysis is identification of dynamic lysine methylation during biological processes or in response to inhibition of a KMT or lysine demethylase. To determine whether this approach can be used to identify candidate methyltransferase substrates in their cellular context we treated 293T cells with UNC0638, a potent and selective inhibitor of the KMTs G9a and GLP (Vedadi et al. (2011) Nat. Chem. Biol. 7, 566-574). We confirmed that in cells treated with the drug, levels of the G9a target H3K9me2 were reduced (FIG. 6A). Triple SILAC labeling coupled with quantitative mass spectrometry was then used to identify changes induced upon drug treatment among the proteins specifically captured by 3×MBT from cytoplasmic, nuclear and chromatin fractions (see schematic, FIG. 6B).

First, the analysis identified an additional 370 proteins enriched by 3×MBT compared to 3×MBT_{D355N} in two biological replicates (Table 5, FIG. 10A). These proteins are strongly enriched for functions in mRNA splicing, translation and cell cycle (FIG. 10B), and they span the cytoplasmic and nuclear compartments (FIG. 10C). Among enriched proteins were 23 candidate G9a/GLP substrates showing reduced association with 3×MBT in response to UNC0638 treatment (Table 1, Table 5). A known G9a substrate, the protein WIZ, has one of the highest scores in this analysis (Rathert et al. (2008) Nat. Chem. Biol. 4, 344-346) (FIGS. 6C-6D, Table 1). Among novel candidate substrate proteins, DNA ligase 1 (LIG1) scores especially strongly and contains the amino acid sequence ARKT, an optimal target site for G9a (Rathert et al., supra) (FIG. 6E, Table 1). Of the 23 candidate substrates, the majority are not present or intact on ProtoArrays, though two hits, MTA1 and RBM15 showed G9a-dependent signal on the protein arrays (FIG. 10E).

Multiple methylation sites present on H3 besides K9 (e.g. H3K4) are not perturbed in response to UNC0638 treatment (Vedadi et al., supra). Thus, the amount of H3 captured by 3×MBT does not decrease appreciably in our analysis and the specific peptide containing K9 after trypsin digestion is not amenable to LC-MS/MS analysis. In addition, we did not reliably detect peptides from other potential G9a substrates, such as SIRT1 (see above) or G9a itself (Rathert et al., supra) due to limitations in the sensitivity and dynamic range of the mass spectrometer—a limitation of available technology and not inherent to 3×MBT as a proteomic tool. However, direct investigation of low abundance peptides identified that 3×MBT capture of a peptide from ACIN1, a known G9a substrate (Rathert et al., supra), was decreased strongly following inhibitor treatment (FIG. 10D). Taken together, the combination of the 3×MBT strategy, SILAC-based quantitative mass spectrometry, and chemical inhibition of KMTs, has allowed us to identify many known and candidate physiologic substrates of G9a and GLP, and argues that such an approach can generally be used to study lysine methylation dynamics at a proteomic level (see Discussion).

Direct Identification of Methylated Proteins

Proteins identified in our 3×MBT pull-down experiments are being enriched in a methyl-dependent fashion, indicating that they are either directly methylated or in a complex containing a methylated protein(s). To determine specific methylation sites, we analyzed the LC-MS/MS data for peptides directly showing methylation of enriched proteins. 102 modified peptides were detected within the 3×MBT pull-down experiments using MaxQuant software. However, automated identification of methylated lysine by MS/MS is particularly difficult because the mass shift for methylation is identical to the difference between certain pairs of amino acids (e.g. glycine and alanine) Therefore to rule out any potential MaxQuant-indicated false positives we manually verified the MS/MS spectra from the 102 potentially modified peptides (see Supplemental Experimental Procedures) (Nichols and White (2009) Methods Mol. Biol. 492, 143-160). We also conducted an independent 3×MBT pull-down from cells metabolically labeled with D3¹³C-L-methionine. The isotopic label adds 4 Dalton to post-translational methylation, giving it a distinctive mass shift. We identified a total of 26 lysine residues that are unambiguously methylated using strict criteria set to rule out any potential false positives: 18 as mono-methyl, 3 as di-methyl and 5 as both mono and di-methyl (Table 2). In every case the highest scoring match for each methylated peptide was observed with the isotopic label corresponding to capture by 3×MBT as opposed to 3×MBT_{D355N}. Notably, twenty-one of the twenty-six methylated residues have not been previously reported. Many additional real methylation events on proteins identified following pull-down with 3×MBT are almost certainly present in our data and could be identified by additional approaches (see Discussion). Taken together, our results highlight the power of using native methyl-lysine-binding domains as affinity reagents in combination with quantitative mass spectrometry to reveal novel methylation events.

Discussion

Lysine methylation is a modification that has been extensively characterized on histone proteins, but about which relatively little is known in the context of non-histone proteins (Su and Tarakhovsky (2006) Curr. Opin. Immunol. 18, 152-157). Notably, there are more than 50 candidate KMTs and 25 KDMTs in the human genome, and the majority of these enzymes are implicated in the etiology of diverse human diseases (Greer and Shi (2012) Nat. Rev. Genet. 13, 343-357; Kooistra and Helin (2012) Nat. Rev. Mol. Cell Biol. 13, 297-311; Petrossian and Clarke (2011) Mol Cell Proteomics 10, M110.000976; Varier and Timmers (2011) Biochim. Biophys. Acta 1815, 75-89). However, the physiologic substrate specificities for many of these KMTs have yet to be discovered, highlighting the need for proteomic strategies to identify novel methylated proteins (Luo (2012) ACS Chem. Biol. 7, 443-463). Prior to our work, a robust method to characterize lysine methylation on a proteome-wide scale had not been reported. Traditional proteomic approaches have been unable to address these questions because of the difficulty in developing pan-specific antibodies for methylated lysine that have the high selectivity and broad specificity required for proteome-wide analysis. Bioinformatics analysis of high-throughput proteomics dataset has identified methylation occurring on a number of abundant proteins in yeast (Pang et al. (2010) BMC Genomics 11, 92), further supporting the notion that methylation is a common regulatory modification and complementing the strategy described here to experimentally enrich methylated lysine.

In the present study we have shown that the naturally occurring 3×MBT domain from L3MBTL1 can be engineered as a general affinity reagent recognizing mono- and di-methylated lysine. The methyl selectivity and lack of sequence specificity of the 3×MBT domain, along with the point mutant negative control, are well-suited for proteome-wide enrichment of protein complexes containing methylated lysine. We have used 3×MBT to detect several known methylation events in vitro and in cells, and to conduct the first proteome-wide enrichment of methylated lysine. Global analysis of proteins and protein complexes carrying lysine methylation shows that this PTM occurs widely across the proteome and is likely involved in a broad range of biological processes. Further, we applied the characterization of cellular lysine methylomes to demonstrate how this strategy can be combined with a chemical biological approach to identify new candidate substrates, such as DNA ligase 1, of the KMTs G9a and GLP. Importantly, we identified known G9a substrates, including WIZ and ACIN1 (Rathert et al., supra). However, a change in overall mono- and di-methylation of histone H3 was not seen. This highlights the constraint that total methylation may not change appreciably when a protein is methylated at high levels on multiple lysine residues or by several KMTs. Regardless, the proof-of-concept strategy demonstrated here for G9a/GLP can be applied to elucidate physiologic substrates of the numerous known and orphan lysine methyltransferases and demethylases present throughout eukaryotic genomes.

The 3×MBT approach presented here complements existing strategies for studying lysine methylation, such as radioactive labeling of the methyl moiety and use of methylation-specific antibodies. The 3×MBT domain can be reproducibly expressed in E. coli, and production of the domain from a recombinant plasmid allows epitope tags and detection strategies to be quickly exchanged, increasing the functionality of the domain when a particular tag is incompatible with the rest of the experimental design.

Though the domain does not appear to have a sequence preference for the amino acids surrounding the methylated lysine, we have observed that it does appear to require the presence of several flanking residues for efficient binding (K. Moore, S. Carlson, and O. Gozani, unpublished observations). This prevents our approach from being applied to trypsin-digested peptides, rather than proteins, in the manner often used for proteomic analysis of modifications such as acetylated lysine (Choudhary et al. (2009) Science 325, 834-840) and phosphorylated tyrosine (Zhang et al. (2005) Mol. Cell Proteomics 4, 1240-1250). We postulate that alternative strategies for protein digestion may generate longer peptides that are more suitable for direct capture by 3×MBT, allowing direct identification of more methylated residues. We envision that the use of native broad-specificity methyl-lysine binding domains can be expanded beyond the 3×MBT of L3MBTL1 to provide new tools to distinguish between mono- and di-methylated lysine, and to study tri-methylated lysine. For example, the 3×MBT domain can be engineered to be selective for binding to only mono-methylated lysine or only di-methylated lysine (Nady et al., supra). Specifically, Nady et al. found that mutating threonine 411 to glutamine results in a preference for monomethyl lysine, while mutating leucine 361 to phenylalanine results in a preference for dimethyl lysine. In addition, multiple domains could be combined to generate “poly” domain reagents with defined or expanded specificity; this approach might be most helpful for detection of tri-methyated protein as we have yet to identify a highly sequence promiscuous trimethyl-lysine binding domain (K. Moore, S. Carlson, and O. Gozani, unpublished observations).

The 3×MBT domain represents a new tool for rapid analysis of methylated lysine. It can be applied to any organism or biological system, and will be applicable to measurement of lysine methylation dynamics during biological processes or resulting from perturbations such as inhibition, knock-down or over-expression of a KMT. Global proteomic analysis will be a powerful tool to investigate the biological function of lysine methyltransferases and demethylases, especially in the many cases where physiological targets of these enzymes have not yet been determined. Quantitative studies will help to integrate lysine methylation into broader signaling networks and contribute to a fuller understanding of how protein lysine methylation regulates diverse cellular processes.

TABLE 1
Proteomic identification of candidate G9a/GLP substrates. 23 proteins are
enriched by 3xMBT compared to 3xMBT_{D355N,} and show decreased
association with 3xMBT following treatment of cells with the inhibitor
UNC0638.

| | Protein Ratios | |
|---|---|---|
| | 3xMBT/ | DMSO/ |
| Protein | D355N | UNC0638 |
| Splicing regulatory glutamine/lysine-rich protein 1 | 2.9 | 4.4 |
| Cleavage and polyadenylation specificity factor subunit 2 | 8.1 | 3.1 |
| Peroxiredoxin-2 | 8.6 | 2.3 |
| RNA-binding protein 15 | 9.9 | 1.9 |
| DNA ligase 1 | 5.9 | 1.9 |
| Apoptosis inhibitor 5 | 8.6 | 1.6 |
| Hematological and neurological-expressed 1-like protein | 8.9 | 1.6 |
| DDB1- and CUL4-associated factor 7 | 2.8 | 1.5 |
| DNA-dependent protein kinase catalytic subunit | 4.5 | 1.5 |
| Protein Wiz | 2.7 | 1.5 |
| DNA-binding protein A | 11.1 | 1.4 |
| Peptidyl-prolyl cis-trans isomerase-like 4 | 4.0 | 1.4 |
| Metastasis-associated protein 1 | 6.0 | 1.4 |
| ATP-dependent DNA helicase Q1 | 18.8 | 1.4 |
| Cytoplasmic FMR1-interacting protein 1 | 3.4 | 1.4 |
| 60S ribosomal protein L5 | 2.3 | 1.4 |
| Clathrin heavy chain 1 | 6.0 | 1.4 |
| Probable ATP-dependent RNA helicase DDX46 | 5.2 | 1.4 |
| Protein syndesmos | 3.5 | 1.3 |
| Probable ATP-dependent RNA helicase DDX23 | 4.8 | 1.3 |
| SWI/SNF-related matrix-associated actin-dependent | 5.8 | 1.3 |
| regulator of chromatin subfamily A member 5 | | |
| Heterogeneous nuclear ribonucleoprotein L | 2.4 | 1.3 |
| WD repeat-containing protein 70 | 5.9 | 1.3 |

TABLE 2
Methylated residues directly identified in 3xMBT pull-down experiments. 29
methylation events at 24 lysine residues are identified with high confidence from 3xMBT
pull-down using by automated analysis followed by manual validation. Reported
methylation sites appear in the NCBI protein database (pubmed.org) or PhosphoSitePlus
(phosphosite.org) as of Nov. 12, 2012. Multiple proteins are listed if the same
peptide occurs in more than one context. H3 and NRK do not have ratios because they
were only identified in the methionine-labeling experiment, MS1 signal for ERC1/2 was
too low to be reliably quantified.

| | Residue | | Protein Ratio | |
|---|---|---|---|---|
| Protein | (methylation state) | | 3xMBT/D355N | Reported |
| CMAS | 399 | (mono) | 4.2 | No |
| DDX1 | 234 | (mono) | 11.2 | No |
| eEF1A1/eEF1A2 | 55 | (mono, di) | 5.2/1.5 | Yes |
| eEF1A1 | 165 | (mono, di) | 5.2 | Yes |
| | 384 | (di) | | Yes |
| | 408 | (mono) | | No |
| eEF-2 | 594 | (mono) | 4.4 | No |
| ERC1/2 | 319/315 | (di) | — | No |
| FAM50A | 5 | (mono) | 3.7* | No |
| GTF2I | 219 | (mono) | 1.5 | No |
| H3 | 79 | (mono, di) | — | Yes |
| hnRNP D/hn RNP A/B | 129 | (mono) | 22.5/13.4 | No |
| hnRNP K | 139 | (mono) | 8.0 | No |
| LIG1 | 795 | (mono) | 10.7 | No |
| MCM4 | 216 | (mono) | 6.6 | No |
| MCM7 | 449 | (mono, di) | 6.7 | No |
| MSH6 | 1126 | (mono) | 1.2 | No |
| NHP2L1 | 33 | (mono) | 4.4* | No |
| NRK | 818 | (di) | — | No |
| RBM25 | 97 | (mono) | 7.7 | No |
| TD-60 | 293 | (mono) | 1.7 | No |
| SAFB1/SAFB2 | 226/225 | (mono) | 9.9 | No |
| SCML2 | 123 | (mono) | 1.7 | No |
| SSRP1 | 143 | (mono) | 14.6 | No |
| TUBB2C/TUBB4† | 19 | (mono) | 2.1/1.9 | No |
| WIZ | 967 | (mono, di) | 5.3 | Yes |

*Protein identified in only one labeling experiment.
†Peptide matches additional proteins, see Table 5.

TABLE 3
related to FIG. 4. Potential G9a substrates identified using 3xMBT to probe ProtoArrays. Protein arrays bearing almost
9500 recombinant human proteins were methylated using GST-tagged recombinant G9a SET domain or GST alone as a control as
shown in FIG. 4. Methylation events were detected using 3xFlag-3xMBT, followed by α-Flag and α-mouse antibodies for
visualization. Results were filtered for proteins scoring a GST array signal-to-noise ratio (SNR) less than or equal to 1.5, a G9a array
SNR greater to or equal to 10, and a difference between the G9a and GST SNRs of greater than or equal to 12. Subsequent proteins
were individually inspected for irregular array features. Hits that fit all of these criteria and were visually validated are shown.

| | | | | | GST Average | G9a Average | G9a- |
|---|---|---|---|---|---|---|---|
| Hit # | Block | Column | Row | Name | SNR | SNR | GST |
| 1 | 16 | 3 | 9 | C2orf43−chromosome 2 open reading frame | −0.147 | 11.92 | 12.067 |
| | | | | 43 (C2orf43) | | | |
| 2 | 22 | 13 | 3 | TMEM32−transmembrane protein 32 | 1.5 | 13.585 | 12.085 |
| | | | | (TMEM32) | | | |
| 3 | 23 | 7 | 8 | ACSL4−acyl-CoA synthetase long-chain family | 1.414 | 13.53 | 12.116 |
| | | | | member 4 (ACSL4), transcript variant 2 | | | |
| 4 | 35 | 5 | 20 | CNTD1−Cyclin N-terminal domain-containing | 0.567 | 12.7485 | 12.1815 |
| | | | | protein 1 | | | |
| 5 | 42 | 7 | 17 | CA13−carbonic anhydrase XIII (CA13) | 0.7275 | 12.9575 | 12.23 |
| 6 | 48 | 11 | 12 | C12orf23−chromosome 12 open reading | 0.4805 | 12.716 | 12.2355 |
| | | | | frame 23 (C12orf23) | | | |
| 7 | 2 | 15 | 21 | CTSZ−Cathepsin Z | 1.281 | 13.5965 | 12.3155 |
| 8 | 34 | 1 | 12 | HPCAL4−hippocalcin like 4 (HPCAL4) | −0.871 | 11.4525 | 12.3235 |
| 9 | 30 | 1 | 21 | PPIL5−Peptidylprolyl isomerase-like 5 | −0.512 | 11.8435 | 12.3555 |
| 10 | 25 | 21 | 2 | RHBDD1−rhomboid domain containing 1 | −0.1095 | 12.264 | 12.3735 |
| | | | | (RHBDD1) | | | |
| 11 | 31 | 1 | 8 | DYNC2LI1−dynein, cytoplasmic 2, light | −0.7785 | 11.6225 | 12.401 |
| | | | | intermediate chain 1, mRNA (cDNA clone | | | |
| | | | | MGC:12166 IMAGE:3828551), complete cds | | | |
| 12 | 21 | 9 | 15 | TGDS−TDP-glucose 4,6-dehydratase (TGDS) | 1.1485 | 13.569 | 12.4205 |
| 13 | 42 | 21 | 18 | C12orf60−Uncharacterized protein C12orf60 | −0.007 | 12.444 | 12.451 |
| 14 | 39 | 1 | 17 | CRYZL1−crystallin, zeta (quinone reductase)- | −1.4165 | 11.172 | 12.5885 |
| | | | | like 1 (CRYZL1) | | | |
| 15 | 29 | 17 | 7 | SH3YL1−SH3 domain containing, Ysc84-like 1 | 0.26 | 12.943 | 12.683 |
| | | | | (S. cerevisiae), mRNA (cDNA clone | | | |
| | | | | MGC:12275 IMAGE:3996120), complete cds | | | |
| 16 | 25 | 15 | 18 | GGPS1−Geranylgeranyl pyrophosphate | −0.4835 | 12.288 | 12.7715 |
| | | | | synthetase | | | |
| 17 | 5 | 13 | 9 | LTC4S−Leukotriene C4 synthase | 1.2085 | 14.074 | 12.8655 |
| 18 | 42 | 11 | 13 | CUTC−cutC copper transporter homolog (E. | 0.6675 | 13.593 | 12.9255 |
| | | | | coli) (CUTC) | | | |
| 19 | 42 | 11 | 5 | SSR4−signal sequence receptor, delta | 1.3285 | 14.355 | 13.0265 |
| | | | | (translocon−associated protein delta) (SSR4) | | | |
| 20 | 31 | 1 | 4 | RAB7B−RAB7B, member RAS oncogene | −0.309 | 12.718 | 13.027 |
| | | | | family (RAB7B) | | | |
| 21 | 9 | 11 | 13 | GAL−galanin prepropeptide (GAL) | −0.0625 | 13.126 | 13.1885 |
| 22 | 5 | 19 | 8 | CDK10−cyclin-dependent kinase (CDC2-like) | 0.3665 | 13.5795 | 13.213 |
| | | | | 10 (CDK10), transcript variant a | | | |
| 23 | 11 | 1 | 7 | HS3ST1−heparan sulfate (glucosamine) 3-O- | 0.092 | 13.3205 | 13.2285 |
| | | | | sulfotransferase 1 (HS3ST1) | | | |
| 24 | 23 | 21 | 5 | PPARG−peroxisome proliferator-activated | −0.4365 | 12.817 | 13.2535 |
| | | | | receptor gamma (PPARG), transcript variant 4 | | | |
| 25 | 29 | 17 | 8 | GYPC−glycophorin C (Gerbich blood group) | 0.081 | 13.3755 | 13.2945 |
| | | | | (GYPC), transcript variant 2 | | | |
| 26 | 15 | 3 | 8 | SEC22B−Vesicle-trafficking protein SEC22b | −0.7975 | 12.6285 | 13.426 |
| 27 | 25 | 19 | 17 | ZRANB1−zinc finger, RAN-binding domain | −0.4305 | 13.164 | 13.5945 |
| | | | | containing 1 (ZRANB1) | | | |
| 28 | 33 | 21 | 8 | NME7−non−metastatic cells 7, protein | 0.543 | 14.146 | 13.603 |
| | | | | expressed in (nucleoside-diphosphate kinase) | | | |
| | | | | (NME7), transcript variant 2 | | | |
| 29 | 33 | 17 | 2 | KCTD7−potassium channel tetramerisation | 1.092 | 14.744 | 13.652 |
| | | | | domain containing 7 (KCTD7) | | | |
| 30 | 29 | 5 | 9 | CMTM3−CKLF-like MARVEL transmembrane | 0.469 | 14.137 | 13.668 |
| | | | | domain containing 3 (CMTM3), transcript | | | |
| | | | | variant 4 | | | |
| 31 | 15 | 5 | 12 | FKBP14−FK506 binding protein 14, 22 kDa | 1.2415 | 14.9685 | 13.727 |
| | | | | (FKBP14) | | | |
| 32 | 38 | 17 | 21 | SCGB2A2−Mammaglobin-A | 0.983 | 14.7535 | 13.7705 |
| 33 | 26 | 7 | 6 | RTN1−reticulon 1 (RTN1), transcript variant 3 | 0.9535 | 14.7335 | 13.78 |
| 34 | 46 | 3 | 10 | STK17B−Serine/threonine-protein kinase 17B | 0.5745 | 14.389 | 13.8145 |
| 35 | 9 | 19 | 7 | LOC145788−PREDICTED: Homo sapiens | 0.224 | 14.056 | 13.832 |
| | | | | hypothetical LOC145788 (LOC145788) | | | |
| 36 | 27 | 1 | 6 | PSMA8−proteasome (prosome, macropain) | −0.8455 | 13.1015 | 13.947 |
| | | | | subunit, alpha type, 8 (PSMA8) | | | |
| 37 | 19 | 1 | 6 | ZBP1−Z-DNA binding protein 1 (ZBP1) | 0.628 | 14.6625 | 14.0345 |
| 38 | 10 | 7 | 19 | MOBKL3−Mps one binder kinase activator- | −0.4505 | 13.5875 | 14.038 |
| | | | | like 3 | | | |
| 39 | 30 | 11 | 14 | NME1−non-metastatic cells 1, protein | 1.0615 | 15.1825 | 14.121 |
| | | | | (NM23A) expressed in (NME1), transcript | | | |
| | | | | variant 2 | | | |
| 40 | 30 | 15 | 7 | CDK5RAP3−CDK5 regulatory subunit- | −0.3395 | 13.8045 | 14.144 |
| | | | | associated protein 3 | | | |
| 41 | 29 | 21 | 2 | YPEL5−yippee-like 5 (Drosophila) (YPEL5) | −0.7875 | 13.404 | 14.1915 |
| 42 | 34 | 21 | 4 | DGKA−diacylglycerol kinase, alpha 80 kDa | 1.2255 | 15.4785 | 14.253 |
| | | | | (DGKA) | | | |
| 43 | 38 | 17 | 8 | C12orf62−chromosome 12 open reading | 0.575 | 14.887 | 14.312 |
| | | | | frame 62 (C12orf62) | | | |
| 44 | 48 | 1 | 9 | MGC10433−RNA-binding protein 42 | −0.3575 | 14.1075 | 14.465 |
| 45 | 25 | 17 | 14 | CAB39L−Calcium-binding protein 39-like | 0.1825 | 14.6485 | 14.466 |
| 46 | 26 | 1 | 22 | CD79A−B-cell antigen receptor complex- | −0.746 | 13.7655 | 14.5115 |
| | | | | associated protein alpha-chain | | | |
| 47 | 30 | 7 | 8 | GPX1−glutathione peroxidase 1 (GPX1) | 1.2215 | 15.7525 | 14.531 |
| 48 | 38 | 1 | 6 | RASSF4−Ras association (RaIGDS/AF-6) | 1.1565 | 15.711 | 14.5545 |
| | | | | domain family 4 (RASSF4) | | | |
| 49 | 15 | 21 | 11 | C10orf83−chromosome 10 open reading | 0.742 | 15.3735 | 14.6315 |
| | | | | frame 83 (C10orf83) | | | |
| 50 | 35 | 1 | 20 | C11orf73−chromosome 11 open reading | −1.399 | 13.508 | 14.907 |
| | | | | frame 73 (C11orf73) | | | |
| 51 | 29 | 19 | 9 | Magmas−mitochondria-associated protein | −1.1595 | 13.928 | 15.0875 |
| | | | | involved in granulocyte-macrophage colony- | | | |
| | | | | stimulating factor signal transduction | | | |
| | | | | (Magmas), nuclear gene encoding | | | |
| | | | | mitochondrial protein | | | |
| 52 | 46 | 3 | 9 | BHMT−betaine-homocysteine | 1.4365 | 16.6285 | 15.192 |
| | | | | methyltransferase (BHMT) | | | |
| 53 | 7 | 11 | 9 | WDR45−WD repeat domain 45 (WDR45), | 1.346 | 16.5645 | 15.2185 |
| | | | | transcript variant 1 | | | |
| 54 | 23 | 17 | 6 | TCTA−T-cell leukemia translocation altered | 1.175 | 16.3955 | 15.2205 |
| | | | | gene (TCTA) | | | |
| 55 | 12 | 11 | 13 | GNPTAB−N-acetylglucosamine-1-phosphate | 1.148 | 16.4795 | 15.3315 |
| | | | | transferase, alpha and beta subunits | | | |
| | | | | (GNPTAB) | | | |
| 56 | 34 | 19 | 7 | AP1S1−adaptor-related protein complex 1, | 1.199 | 16.577 | 15.378 |
| | | | | sigma 1 subunit (AP1S1), transcript variant 1 | | | |
| 57 | 18 | 21 | 7 | CRB3−crumbs homolog 3 (Drosophila) | −0.25 | 15.2185 | 15.4685 |
| | | | | (CRB3), transcript variant 2 | | | |
| 58 | 42 | 1 | 12 | TMED5−transmembrane emp24 protein | −0.425 | 15.0765 | 15.5015 |
| | | | | transport domain containing 5 (TMED5) | | | |
| 59 | 45 | 19 | 8 | VPS29−vacuolar protein sorting 29 homolog | 1.087 | 16.5985 | 15.5115 |
| | | | | (S. cerevisiae) (VPS29), transcript variant 1 | | | |
| 60 | 17 | 21 | 13 | LYPLAL1−lysophospholipase-like 1 (LYPLAL1) | 0.066 | 15.699 | 15.633 |
| 61 | 27 | 13 | 21 | GFOD1−Glucose-fructose oxidoreductase | 0.413 | 16.395 | 15.982 |
| | | | | domain-containing protein 1 | | | |
| 62 | 38 | 21 | 8 | C10orf58−chromosome 10 open reading | 0.966 | 16.988 | 16.022 |
| | | | | frame 58 (C10orf58) | | | |
| 63 | 42 | 17 | 8 | TEAD4−TEA domain family member 4 | 0.753 | 16.8855 | 16.1325 |
| | | | | (TEAD4), transcript variant 3 | | | |
| 64 | 42 | 5 | 21 | HIC2−Hypermethylated in cancer 2 protein | 0.5875 | 16.7275 | 16.14 |
| 65 | 30 | 13 | 14 | SH3YL1−SH3 domain containing, Ysc84-like 1 | 0.3765 | 16.531 | 16.1545 |
| | | | | (S. cerevisiae) (SH3YL1) | | | |
| 66 | 30 | 21 | 14 | SMYD3−SET and MYND domain containing 3 | 1.3045 | 17.5725 | 16.268 |
| | | | | (SMYD3) | | | |
| 67 | 27 | 3 | 6 | PAEP−progestagen-associated endometrial | 0.998 | 17.5 | 16.502 |
| | | | | protein (placental protein 14, pregnancy- | | | |
| | | | | associated endometrial alpha-2-globulin, | | | |
| | | | | alpha uterine protein), transcript variant 2 | | | |
| | | | | (PAEP) | | | |
| 68 | 27 | 1 | 8 | LARP6−La ribonucleoprotein domain family, | −1.032 | 15.594 | 16.626 |
| | | | | member 6 (LARP6), transcript variant 2 | | | |
| 69 | 43 | 1 | 15 | C1QTNF6−C1q and tumor necrosis factor | −0.3795 | 16.3815 | 16.761 |
| | | | | related protein 6 (C1QTNF6), transcript | | | |
| | | | | variant 1 | | | |
| 70 | 15 | 17 | 14 | LYRM7−Lyrm7 homolog (mouse) (LYRM7) | 0.278 | 17.173 | 16.895 |
| 71 | 12 | 3 | 8 | SNFT−Jun dimerization protein p21SNFT | 1.047 | 18.149 | 17.102 |
| | | | | (SNFT) | | | |
| 72 | 35 | 17 | 13 | SERPINB2−serpin peptidase inhibitor, clade B | 0.54 | 17.8615 | 17.3215 |
| | | | | (ovalbumin), member 2 (SERPINB2) | | | |
| 73 | 39 | 3 | 8 | RPL14−60S ribosomal protein L14 | 0.7275 | 18.4075 | 17.68 |
| 74 | 31 | 17 | 18 | CSF3−Granulocyte colony-stimulating factor | 1.2705 | 18.9825 | 17.712 |
| 75 | 27 | 13 | 12 | N/A−cDNA clone IMAGE:4623222, partial cds | 1.0355 | 18.843 | 17.8075 |
| 76 | 28 | 3 | 10 | CDKL5−Cyclin-dependent kinase-like 5 | 1.4255 | 19.679 | 18.2535 |
| 77 | 33 | 17 | 16 | SCGB1A1−secretoglobin, family 1A, member | 1.162 | 19.5755 | 18.4135 |
| | | | | 1 (uteroglobin) (SCGB1A1) | | | |
| 78 | 35 | 3 | 12 | CALN1−calneuron 1 (CALN1), transcript | −0.055 | 18.581 | 18.636 |
| | | | | variant 1 | | | |
| 79 | 25 | 21 | 11 | PTCD2−pentatricopeptide repeat domain 2 | 0.6795 | 19.397 | 18.7175 |
| | | | | (PTCD2) | | | |
| 80 | 38 | 17 | 7 | CTXN1−cortexin 1 (CTXN1) | 1.002 | 19.749 | 18.747 |
| 81 | 30 | 7 | 17 | ZNF695−zinc finger protein 695 (ZNF695) | 1.5 | 20.341 | 18.841 |
| 82 | 30 | 5 | 14 | SAR1B−SAR1 gene homolog B (S. cerevisiae) | 0.5585 | 19.833 | 19.2745 |
| | | | | (SAR1B), transcript variant 2 | | | |
| 83 | 42 | 21 | 8 | SMYD5−SMYD family member 5 (SMYD5) | 1.158 | 20.66 | 19.502 |
| 84 | 48 | 17 | 4 | GPR108−G protein-coupled receptor 108 | −0.7885 | 19.218 | 20.0065 |
| | | | | (GPR108) | | | |
| 85 | 20 | 5 | 7 | SMAP1−stromal membrane-associated | 1.37 | 21.532 | 20.162 |
| | | | | protein 1 (SMAP1) | | | |
| 86 | 15 | 11 | 21 | TTC27−Tetratricopeptide repeat protein 27 | 0.496 | 20.663 | 20.167 |
| 87 | 48 | 1 | 7 | RAB33B−RAB33B, member RAS oncogene | 0.0035 | 20.189 | 20.1855 |
| | | | | family (RAB33B) | | | |
| 88 | 34 | 21 | 2 | PHF20L1−PHD finger protein 20-like 1 | 1.1345 | 21.5085 | 20.374 |
| | | | | (PHF20L1), transcript variant 3 | | | |
| 89 | 38 | 21 | 14 | ARL6IP1−ADP-ribosylation factor-like 6 | 1.049 | 21.8215 | 20.7725 |
| | | | | interacting protein 1 (ARL6IP1) | | | |
| 90 | 2 | 17 | 16 | MGC4473−hypothetical protein MGC4473 | 0.0125 | 21.696 | 21.6835 |
| | | | | (MGC4473) | | | |
| 91 | 35 | 17 | 8 | MRPL21−mitochondrial ribosomal protein | 1.412 | 23.2715 | 21.8595 |
| | | | | L21 (MRPL21), nuclear gene encoding | | | |
| | | | | mitochondrial protein, transcript variant 1 | | | |
| 92 | 38 | 1 | 19 | TMEM97−transmembrane protein 97 | −1.5345 | 21.2275 | 22.762 |
| | | | | (TMEM97) | | | |
| 93 | 33 | 11 | 13 | GHRL−ghrelin/obestatin preprohormone | 0.4015 | 24.48 | 24.0785 |
| | | | | (GHRL) | | | |
| 94 | 35 | 21 | 18 | UBE2S−Ubiquitin-conjugating enzyme E2 S | 0.2835 | 24.986 | 24.7025 |
| 95 | 37 | 17 | 8 | CLDN15−claudin 15 (CLDN15) | −0.2965 | 25.003 | 25.2995 |
| 96 | 6 | 9 | 4 | BLCAP−bladder cancer associated protein | 0.9325 | 26.4415 | 25.509 |
| | | | | (BLCAP) | | | |
| 97 | 42 | 3 | 21 | GPX5−glutathione peroxidase 5 (epididymal | −0.0425 | 25.536 | 25.5785 |
| | | | | androgen−related protein) (GPX5), transcript | | | |
| | | | | variant 2, mRNA. | | | |
| 98 | 35 | 1 | 3 | CDC2−cell division cycle 2, G1 to S and G2 to | −0.696 | 25.322 | 26.018 |
| | | | | M (CDC2), transcript variant 1 | | | |
| 99 | 27 | 1 | 3 | RDH11−retinol dehydrogenase 11 (all- | 0.0685 | 26.569 | 26.5005 |
| | | | | trans/9-cis/11-cis) (RDH11) | | | |
| 100 | 41 | 21 | 12 | DIRAS1−DIRAS family, GTP-binding RAS-like 1 | 0.9875 | 29.1755 | 28.188 |
| | | | | (DIRAS1) | | | |
| 101 | 35 | 3 | 8 | LOC400500−PREDICTED: Homo sapiens | −0.1195 | 29.007 | 29.1265 |
| | | | | hypothetical LOC400500 (LOC400500) | | | |
| 102 | 15 | 1 | 12 | KRR1−KRR1, small subunit (SSU) processome | 0.5655 | 29.842 | 29.2765 |
| | | | | component, homolog (yeast) (KRR1) | | | |
| 103 | 15 | 3 | 12 | LRRC20−leucine rich repeat containing 20 | 1.3985 | 32.2875 | 30.889 |
| | | | | (LRRC20) | | | |
| 104 | 25 | 21 | 14 | FTH1−ferritin, heavy polypeptide 1 (FTH1) | −0.8025 | 30.4035 | 31.206 |
| 105 | 11 | 19 | 7 | LPAL2−lipoprotein, Lp(a)-like 2 (LPAL2), | 0.282 | 33.257 | 32.975 |
| | | | | transcript variant 1 | | | |
| 106 | 46 | 3 | 5 | FTL−ferritin, light polypeptide (FTL) | 0.717 | 34.1715 | 33.4545 |
| 107 | 44 | 13 | 21 | CCL24−C-C motif chemokine 24 | 0.109 | 34.513 | 34.404 |
| 108 | 42 | 1 | 18 | UBE2D2−Ubiquitin-conjugating enzyme E2 | −0.383 | 37.2325 | 37.6155 |
| | | | | D2 | | | |
| 109 | 11 | 21 | 18 | FTH1−Ferritin heavy chain | 0.8145 | 38.6675 | 37.853 |
| 110 | 27 | 1 | 15 | MAGEH1−melanoma antigen family H, 1 | 1.114 | 44.0495 | 42.9355 |
| | | | | (MAGEH1) | | | |
| 111 | 11 | 1 | 12 | DNTT−deoxynucleotidyltransferase, terminal | 0.378 | 49.856 | 49.478 |
| | | | | (DNTT), transcript variant 1 | | | |
| 112 | 42 | 21 | 13 | KIFC1−kinesin family member C1 (KIFC1) | 0.7445 | 63.191 | 62.4465 |

TABLE 4
related to FIG. 5. Functional analysis of proteins enriched by 3xMBT. Many Gene Ontology (GO) terms, especially
related to RNA processing, are over-represented among proteins enriched at 2-fold by 3xMBT relative to 3xMBT_{D355N}
from the experiment shown in FIG. 5. Enrichment analysis was performed using DAVID with proteins identified from input
material as the background gene set. GO terms are included if they are enriched with Bonferroni-corrected p-value <0.05.

| | | | | Bonferroni |
|---|---|---|---|---|
| GO Category | GO Term | Count | Enrichment | p-value |
| Molecular | GO:0000166~nucleotide binding | 71 | 1.5 | 2.74E−04 |
| Function | GO:0003723~RNA binding | 64 | 1.7 | 1.02E−05 |
| | GO:0003677~DNA binding | 49 | 1.9 | 1.21E−05 |
| | GO:0042623~ATPase activity, coupled | 17 | 2.5 | 0.036 |
| | GO:0008026~ATP-dependent helicase activity | 15 | 3.4 | 7.20E−04 |
| | GO:0070035~purine NTP-dependent helicase | 15 | 3.4 | 7.20E−04 |
| | activity | | | |
| | GO:0004386~helicase activity | 20 | 3.7 | 9.24E−07 |
| | GO:0003678~DNA helicase activity | 10 | 4.0 | 0.011 |
| | GO:0003724~RNA helicase activity | 9 | 4.3 | 0.014 |
| Cellular | GO:0031974~membrane-enclosed lumen | 81 | 1.7 | 5.07E−09 |
| Component | GO:0070013~intracellular organelle lumen | 81 | 1.7 | 2.71E−09 |
| | GO:0043233~organelle lumen | 81 | 1.7 | 2.71E−09 |
| | GO:0005730~nucleolus | 37 | 1.8 | 0.007 |
| | GO:0031981~nuclear lumen | 81 | 1.9 | 2.73E−12 |
| | GO:0030529~ribonucleoprotein complex | 57 | 1.9 | 8.83E−07 |
| | GO:0005654~nucleoplasm | 62 | 2.1 | 3.75E−11 |
| | GO:0044451~nucleoplasm part | 35 | 2.5 | 4.69E−07 |
| | GO:0016607~nuclear speck | 13 | 2.9 | 0.046 |
| | GO:0016604~nuclear body | 18 | 2.9 | 0.001 |
| | GO:0005681~spliceosome | 40 | 3.7 | 6.49E−17 |
| Biological Process | GO:0045449~regulation of transcription | 40 | 1.7 | 0.043 |
| | GO:0006350~transcription | 39 | 2.1 | 1.48E−04 |
| | GO:0006396~RNA processing | 68 | 2.4 | 9.45E−16 |
| | GO:0008380~RNA splicing | 60 | 2.8 | 1.01E−16 |
| | GO:0000398~nuclear mRNA splicing, via | 42 | 2.8 | 3.02E−10 |
| | spliceosome | | | |
| | GO:0000377~RNA splicing, via | 42 | 2.8 | 3.02E−10 |
| | transesterification reactions with bulged | | | |
| | adenosine as nucleophile | | | |
| | GO:0000375~RNA splicing, via | 42 | 2.8 | 3.02E−10 |
| | transesterification reactions | | | |
| | GO:0016071~mRNA metabolic process | 67 | 2.9 | 1.43E−20 |
| | GO:0006397~mRNA processing | 64 | 2.9 | 7.13E−20 |

TABLE 5
related to FIG. 6. Candidate G9a/GLP substrates identified in cells through
quantitative analysis of dynamic lysine methylation after treatment with UNC0638.
Proteins and SILAC ratios for proteins identified in the pair of 3-channel SILAC
experiments shown in FIG. 6, using cells in light/heavy SILAC media treated with
DMSO/UNC0638 followed by pull-down with 3xMBT, and medium media treated
with DMSO for pull-down with 3xMBT_{D355N}. All values in log base 2.

| | 3xMBT/D355N | | | | | Candidate |
|---|---|---|---|---|---|---|
| Gene | UNC | UNC | Relative to UNC0638 | | | G9a/GLP |
| Symbol | Light | Heavy | UNC Light | UNC Heavy | Average | target |
| HPSE2 | 3.81 | −0.14 | 4.14 | * | 4.14 | |
| NSUN2 | 2.00 | 0.25 | 0.18 | 4.51 | 2.35 | √✓ |
| SFRS12 | 2.39 | 0.65 | 0.27 | 4.00 | 2.13 | ✓ |
| CPSF2 | 4.20 | 1.85 | 3.16 | 0.15 | 1.65 | ✓ |
| PRDX2 | 4.01 | 2.19 | 1.96 | 0.43 | 1.19 | ✓ |
| C20orf43 | 0.71 | 1.26 | −0.46 | 2.48 | 1.01 | |
| RBM15 | 4.48 | 2.14 | 0.26 | 1.65 | 0.96 | ✓ |
| LIG1 | 1.96 | 3.18 | 1.06 | 0.83 | 0.95 | ✓ |
| GTF3C1 | 2.08 | 0.63 | −0.06 | 1.93 | 0.93 | |
| SUPT5H | 1.21 | 3.23 | −0.16 | 1.75 | 0.79 | |
| API5 | 2.67 | 3.54 | 0.31 | 1.12 | 0.71 | ✓ |
| HN1L | 3.57 | 2.73 | 1.22 | 0.17 | 0.70 | ✓ |
| DCAF7 | 1.31 | 1.67 | 0.48 | 0.69 | 0.59 | ✓ |
| PRKDC | 2.39 | 1.96 | 0.95 | 0.21 | 0.58 | ✓ |
| C3orf37 | 1.29 | 1.53 | −0.66 | 1.80 | 0.57 | |
| WIZ | 2.02 | 0.93 | 0.29 | 0.83 | 0.56 | ✓ |
| DNM2 | 0.57 | 0.05 | 0.54 | * | 0.54 | |
| CBR1 | 0.84 | 1.12 | 1.14 | −0.09 | 0.53 | |
| CSDA | 3.10 | 3.85 | 0.73 | 0.30 | 0.52 | ✓ |
| GAPDHS | 1.73 | 3.31 | −2.20 | 3.21 | 0.50 | |
| PPIL4 | 2.38 | 1.63 | 0.50 | 0.46 | 0.48 | ✓ |
| MTA1 | 4.28 | 0.90 | 0.56 | 0.36 | 0.46 | ✓ |
| RECQL | 5.03 | 3.43 | 0.21 | 0.71 | 0.46 | ✓ |
| CYFIP1 | 1.58 | 1.93 | 0.36 | 0.55 | 0.46 | ✓ |
| RPL5 | 1.32 | 1.14 | 0.59 | 0.31 | 0.45 | ✓ |
| CLTC | 2.98 | 2.19 | 0.65 | 0.24 | 0.44 | ✓ |
| DDX46 | 2.67 | 2.10 | 0.33 | 0.53 | 0.43 | ✓ |
| NUDT16L1 | 1.77 | 1.84 | 0.28 | 0.57 | 0.43 | ✓ |
| DDX23 | 2.27 | 2.24 | 0.54 | 0.29 | 0.42 | ✓ |
| SMARCA5 | 2.61 | 2.49 | 0.32 | 0.47 | 0.39 | ✓ |
| HNRNPL | 1.40 | 1.15 | 0.41 | 0.36 | 0.38 | ✓ |
| WDR70 | 2.93 | 2.21 | −0.18 | 0.94 | 0.38 | |
| FAM98A | 4.03 | 2.99 | 0.19 | 0.57 | 0.38 | |
| SEPT7 | 2.54 | 2.75 | 0.26 | 0.50 | 0.38 | |
| RRM1 | 0.94 | 1.23 | −0.22 | 0.96 | 0.37 | |
| KHDRBS1 | 2.82 | 2.03 | 0.19 | 0.54 | 0.37 | |
| PPP1R10 | 2.57 | 4.73 | 0.37 | 0.36 | 0.36 | |
| H2AFX | 2.38 | 2.73 | 0.40 | 0.31 | 0.35 | |
| EFTUD2 | 2.07 | 1.98 | 0.33 | 0.36 | 0.34 | |
| SRP68 | 3.28 | 2.41 | 0.76 | −0.08 | 0.34 | |
| SNRPG | 2.86 | 1.74 | 0.26 | 0.42 | 0.34 | |
| HNRNPH3 | 2.57 | 1.94 | 0.39 | 0.28 | 0.34 | |
| DBR1 | 2.56 | 3.11 | 0.13 | 0.54 | 0.33 | |
| HIST1H2BB | 5.30 | 5.56 | 0.46 | 0.20 | 0.33 | |
| DUT | 2.37 | 1.63 | 0.04 | 0.62 | 0.33 | |
| CTBP2 | 1.74 | 1.97 | 0.15 | 0.50 | 0.32 | |
| HIST2H2BF | 4.76 | 5.02 | 0.45 | 0.19 | 0.32 | |
| CLASP2 | 2.10 | 2.43 | 0.19 | 0.45 | 0.32 | |
| DNPEP | 1.51 | 1.02 | 0.42 | 0.21 | 0.31 | |
| PSME3 | 1.89 | 1.70 | −0.01 | 0.62 | 0.31 | |
| DNAJC2 | 3.04 | 2.18 | 0.33 | 0.28 | 0.30 | |
| HIST1H2AG | 3.76 | 3.54 | 0.38 | 0.23 | 0.30 | |
| NTHL1 | 1.60 | 0.48 | −0.12 | 0.72 | 0.30 | |
| RPS10 | 2.65 | 0.72 | 0.35 | 0.25 | 0.30 | |
| THOC2 | 2.01 | 2.50 | −0.05 | 0.64 | 0.30 | |
| ZNF598 | 3.58 | 1.92 | 0.06 | 0.52 | 0.29 | |
| NUMA1 | 3.33 | 0.83 | −0.32 | 0.90 | 0.29 | |
| PTBP1 | 3.23 | 2.61 | 0.25 | 0.33 | 0.29 | |
| RPL15 | 1.39 | 1.58 | 0.69 | −0.12 | 0.29 | |
| PRPF40A | 2.13 | 3.54 | 0.24 | 0.33 | 0.29 | |
| MTPN | 2.28 | 2.48 | −0.25 | 0.82 | 0.29 | |
| ELAVL1 | 2.42 | 1.93 | 0.36 | 0.21 | 0.29 | |
| RFC4 | 1.80 | 1.95 | 0.26 | 0.32 | 0.29 | |
| RBM3 | 3.25 | 3.49 | 0.33 | 0.24 | 0.29 | |
| CNBP | 1.79 | 1.05 | 0.05 | 0.51 | 0.28 | |
| LARP1 | 4.80 | 4.00 | 0.27 | 0.28 | 0.28 | |
| RPL30 | 2.18 | 1.76 | 0.42 | 0.12 | 0.27 | |
| RPL7A | 1.47 | 1.58 | 0.29 | 0.26 | 0.27 | |
| RBM25 | 2.28 | 2.41 | 0.29 | 0.25 | 0.27 | |
| CAMK2D | 2.82 | 1.96 | 0.27 | 0.26 | 0.27 | |
| POLDIP3 | 2.76 | 2.51 | 0.14 | 0.39 | 0.26 | |
| USP10 | 2.13 | 2.00 | −0.05 | 0.57 | 0.26 | |
| TXN | 1.93 | 1.75 | 0.38 | 0.14 | 0.26 | |
| MATR3 | 1.77 | 1.72 | 0.14 | 0.37 | 0.26 | |
| XRN2 | 2.76 | 2.19 | 0.18 | 0.34 | 0.26 | |
| TRIM25 | 0.76 | 1.88 | 0.46 | 0.05 | 0.25 | |
| SF3B2 | 3.33 | 3.89 | 0.33 | 0.17 | 0.25 | |
| HNRNPH2 | 2.68 | 1.02 | 0.32 | 0.18 | 0.25 | |
| HNRNPU | 2.39 | 2.16 | 0.28 | 0.22 | 0.25 | |
| ZC3HAV1 | 1.92 | 2.03 | 0.65 | −0.16 | 0.25 | |
| PDCD2L | 3.19 | 2.68 | 0.28 | 0.22 | 0.25 | |
| PRDX6 | 2.00 | 2.01 | 0.23 | 0.27 | 0.25 | |
| CACYBP | 1.48 | 1.46 | 0.20 | 0.29 | 0.25 | |
| U2SURP | 2.58 | 3.19 | 0.27 | 0.23 | 0.25 | |
| SNRPC | 3.78 | 3.39 | 0.32 | 0.17 | 0.25 | |
| SEC24A | 2.29 | 1.94 | −0.11 | 0.60 | 0.24 | |
| SRSF11 | 2.22 | 2.45 | 0.34 | 0.15 | 0.24 | |
| CHD4 | 3.63 | 2.24 | 0.14 | 0.34 | 0.24 | |
| MPG | 1.87 | 1.39 | −0.04 | 0.52 | 0.24 | |
| NHP2L1 | 3.46 | 1.56 | 1.33 | −0.86 | 0.24 | |
| CC2D1A | 2.22 | 2.46 | 0.04 | 0.44 | 0.24 | |
| RPL23A | 3.29 | 2.84 | 0.70 | −0.22 | 0.24 | |
| MDC1 | 2.83 | 2.31 | 0.11 | 0.36 | 0.24 | |
| SRSF3 | 2.55 | 2.63 | 0.23 | 0.25 | 0.24 | |
| PEF1 | 1.78 | 1.48 | 0.17 | 0.30 | 0.24 | |
| RCC1 | 2.25 | 2.15 | 0.29 | 0.18 | 0.24 | |
| SRP9 | 1.52 | 1.88 | 0.01 | 0.46 | 0.23 | |
| RUVBL2 | 1.47 | 1.75 | 1.48 | −1.02 | 0.23 | |
| RPLP0 | 2.04 | 1.64 | 0.44 | 0.02 | 0.23 | |
| PPP1CB | 2.55 | 2.35 | 0.18 | 0.28 | 0.23 | |
| CNN3 | 2.35 | 2.36 | −0.15 | 0.61 | 0.23 | |
| MECP2 | 3.59 | 2.74 | 0.27 | 0.19 | 0.23 | |
| SAFB | 2.76 | 2.38 | 0.35 | 0.11 | 0.23 | |
| RPL18 | 1.55 | 0.60 | 0.45 | 0.01 | 0.23 | |
| YWHAE | 2.52 | 1.86 | −0.12 | 0.58 | 0.23 | |
| TUBB4B | 1.75 | 1.05 | 0.03 | 0.42 | 0.23 | |
| HDGF | 2.78 | 1.65 | 0.22 | 0.23 | 0.23 | |
| WDR82 | 2.93 | 3.11 | 0.21 | 0.24 | 0.23 | |
| SNRNP200 | 3.14 | 2.08 | 0.26 | 0.19 | 0.22 | |
| TUBB | 1.42 | 1.16 | 0.14 | 0.31 | 0.22 | |
| RBBP4 | 2.23 | 3.21 | 0.23 | 0.21 | 0.22 | |
| ADAR | 2.47 | 2.00 | 0.07 | 0.37 | 0.22 | |
| HNRNPUL1 | 3.07 | 2.97 | 0.61 | −0.17 | 0.22 | |
| LIG3 | 2.16 | 2.43 | 0.18 | 0.26 | 0.22 | |
| GSK3B | 2.25 | 1.95 | 0.03 | 0.41 | 0.22 | |
| ZC3H11A | 2.81 | 2.23 | 0.12 | 0.32 | 0.22 | |
| CHERP | 2.26 | 1.58 | 0.05 | 0.38 | 0.22 | |
| RFC1 | −1.32 | 1.29 | −0.18 | 0.61 | 0.21 | |
| ZNF207 | 3.12 | 2.48 | 0.09 | 0.34 | 0.21 | |
| HNRNPH1 | 2.27 | 1.85 | 0.19 | 0.24 | 0.21 | |
| H2AFV | 4.71 | 3.32 | 0.23 | 0.19 | 0.21 | |
| RPL6 | 1.87 | 2.29 | 0.11 | 0.31 | 0.21 | |
| TOP2B | 2.04 | 1.61 | 0.21 | 0.21 | 0.21 | |
| RALY | 3.37 | 1.15 | 0.31 | 0.12 | 0.21 | |
| ORC3 | 2.54 | 1.54 | 0.17 | 0.25 | 0.21 | |
| ATXN2L | 2.66 | 2.64 | 0.12 | 0.31 | 0.21 | |
| SEC23B | 3.16 | 3.24 | 0.24 | 0.17 | 0.21 | |
| DDX5 | 2.92 | 3.00 | 0.10 | 0.31 | 0.20 | |
| CRKL | 1.59 | 1.16 | 0.03 | 0.37 | 0.20 | |
| HNRNPAB | 4.21 | 4.00 | 0.32 | 0.09 | 0.20 | |
| HNRPDL | 3.02 | 2.81 | 0.24 | 0.16 | 0.20 | |
| MCM2 | 3.23 | 2.76 | 0.19 | 0.21 | 0.20 | |
| DHX15 | 2.54 | 2.98 | 0.27 | 0.13 | 0.20 | |
| DDX3X | 2.58 | 2.64 | 0.14 | 0.25 | 0.20 | |
| HSPA5 | 2.32 | 0.70 | 0.05 | 0.35 | 0.20 | |
| RPS7 | 2.76 | 2.56 | 0.25 | 0.15 | 0.20 | |
| FOCAD | 2.73 | 1.83 | 0.20 | 0.20 | 0.20 | |
| MCM4 | 2.16 | 1.92 | 0.15 | 0.24 | 0.20 | |
| TARDBP | 2.40 | 2.44 | 0.21 | 0.18 | 0.20 | |
| CPSF6 | 3.10 | 1.89 | 0.09 | 0.30 | 0.20 | |
| SF3B14 | 1.56 | 1.33 | 0.14 | 0.25 | 0.20 | |
| GIGYF2 | 3.04 | 2.62 | 0.05 | 0.34 | 0.19 | |
| TOP1 | 3.87 | 3.47 | 0.22 | 0.16 | 0.19 | |
| RAN | 1.98 | 2.17 | 0.25 | 0.14 | 0.19 | |
| FUS | 2.10 | 2.17 | 0.34 | 0.05 | 0.19 | |
| YBX1 | 2.06 | 1.88 | 0.11 | 0.28 | 0.19 | |
| EIF2S2 | 2.19 | 2.09 | 0.02 | 0.36 | 0.19 | |
| GNB1L | 2.60 | 2.24 | 0.12 | 0.25 | 0.19 | |
| SRP72 | 2.90 | 1.68 | 0.10 | 0.27 | 0.19 | |
| GPX4 | 2.72 | 2.28 | 0.88 | −0.50 | 0.19 | |
| KIF2A | 2.33 | 2.05 | 0.15 | 0.23 | 0.19 | |
| CALD1 | 3.50 | 3.53 | 0.09 | 0.29 | 0.19 | |
| G3BP1 | 2.72 | 2.02 | 0.15 | 0.23 | 0.19 | |
| TFG | 4.03 | 4.21 | 0.22 | 0.16 | 0.19 | |
| VPS35 | 2.80 | 3.05 | 0.25 | 0.12 | 0.19 | |
| SRP14 | 2.96 | 2.23 | 0.07 | 0.30 | 0.19 | |
| RPL31 | 2.82 | 2.08 | 0.51 | −0.14 | 0.19 | |
| SH3GL1 | 2.38 | 1.99 | −0.23 | 0.60 | 0.19 | |
| DIDO1 | 3.36 | 1.35 | 0.37 | 0.00 | 0.18 | |
| DDX39A | 1.53 | 2.00 | 0.01 | 0.36 | 0.18 | |
| LANCL1 | 1.63 | 1.33 | 0.23 | 0.14 | 0.18 | |
| LSM12 | 3.81 | 2.07 | 0.34 | 0.03 | 0.18 | |
| SF3B3 | 2.08 | 2.48 | 0.31 | 0.06 | 0.18 | |
| ILF2 | 3.45 | 3.72 | 0.22 | 0.15 | 0.18 | |
| MCM3 | 2.49 | 2.59 | 0.09 | 0.28 | 0.18 | |
| HNRNPA3 | 3.79 | 2.96 | 0.16 | 0.20 | 0.18 | |
| MCM6 | 2.53 | 2.48 | 0.26 | 0.10 | 0.18 | |
| HCFC1 | 1.69 | 1.25 | −0.18 | 0.54 | 0.18 | |
| DDX6 | 3.05 | 3.18 | 0.22 | 0.14 | 0.18 | |
| HNRNPR | 3.60 | 3.20 | 0.24 | 0.12 | 0.18 | |
| DHX9 | 3.53 | 3.27 | 0.28 | 0.08 | 0.18 | |
| CPSF3 | 0.51 | 3.39 | 0.06 | 0.30 | 0.18 | |
| DAZAP1 | 2.10 | 2.92 | 0.35 | 0.01 | 0.18 | |
| AMOT | 2.44 | 2.95 | 0.39 | −0.03 | 0.18 | |
| EIF4E | 2.40 | 2.40 | 0.31 | 0.04 | 0.18 | |
| MK167 | 2.26 | 2.59 | −0.25 | 0.61 | 0.18 | |
| HNRNPA2B1 | 3.97 | 3.43 | 0.28 | 0.07 | 0.18 | |
| RPS4X | 1.32 | 1.69 | 0.36 | −0.01 | 0.18 | |
| SNX9 | 2.47 | 2.60 | 0.04 | 0.31 | 0.18 | |
| HNRNPD | 4.56 | 4.07 | 0.29 | 0.06 | 0.18 | |
| PRPF4 | 2.26 | 2.85 | −0.28 | 0.63 | 0.18 | |
| EIF4A1 | 3.68 | 3.59 | 0.05 | 0.30 | 0.18 | |
| HSPA8 | 1.48 | 2.05 | 0.06 | 0.28 | 0.17 | |
| NPM1 | 1.27 | 1.24 | 0.11 | 0.24 | 0.17 | |
| U2AF2 | 2.60 | 2.86 | 0.20 | 0.14 | 0.17 | |
| SKP1 | 1.97 | 2.42 | −0.01 | 0.36 | 0.17 | |
| ALYREF | 3.06 | 4.17 | 0.22 | 0.12 | 0.17 | |
| PALLD | 2.31 | 2.50 | 0.09 | 0.26 | 0.17 | |
| HIST1H1E | 3.27 | 3.40 | −0.11 | 0.45 | 0.17 | |
| RBMX | 4.49 | 3.98 | 0.24 | 0.11 | 0.17 | |
| ILF3 | 4.28 | 3.53 | 0.23 | 0.11 | 0.17 | |
| PCBP2 | 2.41 | 1.88 | 0.21 | 0.13 | 0.17 | |
| HNRNPA1 | 4.25 | 3.75 | 0.25 | 0.09 | 0.17 | |
| GRWD1 | 2.55 | 2.87 | 0.09 | 0.24 | 0.17 | |
| HIST1H1C | 4.61 | 5.16 | 0.15 | 0.18 | 0.17 | |
| CTTN | 2.75 | 1.52 | 0.08 | 0.26 | 0.17 | |
| CORO1C | 3.30 | 2.83 | 0.26 | 0.07 | 0.17 | |
| SEPT2 | 2.49 | 1.89 | 0.12 | 0.21 | 0.17 | |
| SFPQ | 3.86 | 3.19 | 0.25 | 0.08 | 0.17 | |
| CMAS | 2.86 | 2.71 | 0.12 | 0.22 | 0.17 | |
| NONO | 3.75 | 3.18 | 0.16 | 0.17 | 0.17 | |
| DDX17 | 2.86 | 2.61 | 0.12 | 0.21 | 0.16 | |
| C14orf166 | 3.13 | 3.09 | 0.24 | 0.09 | 0.16 | |
| DRG1 | 3.66 | 3.49 | −0.03 | 0.36 | 0.16 | |
| PABPC4 | 3.59 | 2.94 | 0.17 | 0.16 | 0.16 | |
| ARID1A | 2.71 | 2.61 | 0.17 | 0.15 | 0.16 | |
| EIF4A3 | 3.11 | 3.44 | 0.10 | 0.23 | 0.16 | |
| KHSRP | 3.18 | 3.34 | 0.16 | 0.17 | 0.16 | |
| PSIP1 | 2.92 | 2.72 | 0.15 | 0.17 | 0.16 | |
| CAPZA1 | 2.01 | 1.83 | 0.06 | 0.26 | 0.16 | |
| EIF4G1 | 3.31 | 3.18 | 0.25 | 0.07 | 0.16 | |
| CPSF1 | 3.15 | 2.44 | 0.15 | 0.17 | 0.16 | |
| EXOSC4 | 1.91 | 3.98 | 0.13 | 0.19 | 0.16 | |
| GTF2I | 2.75 | 3.02 | 0.00 | 0.31 | 0.16 | |
| SNRPD1 | 3.25 | 2.90 | 0.14 | 0.17 | 0.16 | |
| DDX1 | 3.64 | 3.07 | 0.29 | 0.02 | 0.16 | |
| XRCC5 | 2.80 | 2.15 | 0.17 | 0.14 | 0.15 | |
| SYNCRIP | 3.48 | 2.28 | 0.17 | 0.14 | 0.15 | |
| PARP1 | 3.17 | 2.72 | 0.22 | 0.09 | 0.15 | |
| CKAP5 | 1.30 | 2.10 | 0.06 | 0.24 | 0.15 | |
| ZNF326 | 6.59 | 3.01 | 0.33 | −0.02 | 0.15 | |
| EEF1A1 | 2.87 | 3.08 | 0.04 | 0.27 | 0.15 | |
| HNRNPA0 | 3.80 | 2.94 | 0.28 | 0.02 | 0.15 | |
| KIN | 2.11 | 2.85 | −0.04 | 0.35 | 0.15 | |
| C4orf27 | 3.14 | 3.45 | 0.21 | 0.09 | 0.15 | |
| PCBP1 | 2.49 | 2.16 | 0.19 | 0.11 | 0.15 | |
| SRP19 | 2.14 | 1.88 | 0.38 | −0.08 | 0.15 | |
| RPL12 | 1.79 | 1.76 | 0.31 | 0.00 | 0.15 | |
| EEF1D | 3.56 | 3.34 | 0.20 | 0.11 | 0.15 | |
| PABPC1 | 3.44 | 2.91 | 0.26 | 0.04 | 0.15 | |
| ZC3HAV1L | 2.00 | 1.71 | 0.16 | 0.14 | 0.15 | |
| C22orf28 | 2.67 | 2.49 | 0.22 | 0.08 | 0.15 | |
| SNRPA1 | 3.39 | 2.94 | 0.22 | 0.08 | 0.15 | |
| EIF4E2 | 2.26 | 1.89 | 0.06 | 0.24 | 0.15 | |
| BUB3 | 3.26 | 2.75 | 0.14 | 0.15 | 0.15 | |
| RCC2 | 2.89 | 2.61 | 0.26 | 0.03 | 0.15 | |
| RBM26 | 2.00 | 2.44 | 0.08 | 0.21 | 0.14 | |
| DENR | 3.36 | 2.50 | 0.20 | 0.09 | 0.14 | |
| U2AF1 | 2.52 | 3.19 | 0.08 | 0.20 | 0.14 | |
| KPNB1 | 2.72 | 2.58 | 0.06 | 0.23 | 0.14 | |
| SF3B1 | 3.31 | 3.17 | 0.18 | 0.11 | 0.14 | |
| NCL | 2.90 | 2.71 | 0.24 | 0.04 | 0.14 | |
| MTHFD1 | 2.35 | 1.99 | 0.23 | 0.05 | 0.14 | |
| TCOF1 | 3.27 | 0.67 | 0.38 | −0.10 | 0.14 | |
| ALKBH5 | 1.73 | 1.96 | −0.01 | 0.29 | 0.14 | |
| SH3PXD2B | 3.71 | 3.07 | 0.37 | −0.09 | 0.14 | |
| PRPF8 | 2.50 | 2.51 | 0.17 | 0.11 | 0.14 | |
| DSTN | 2.54 | 3.00 | 0.04 | 0.24 | 0.14 | |
| C19orf43 | 3.12 | 2.47 | 0.24 | 0.03 | 0.14 | |
| SNRPD3 | 3.20 | 3.95 | 0.27 | 0.01 | 0.14 | |
| SNRPA | 5.10 | 4.73 | 0.11 | 0.16 | 0.14 | |
| DDX21 | 1.00 | 0.55 | 0.14 | * | 0.14 | |
| SNRPB | 5.48 | 5.39 | 0.30 | −0.03 | 0.14 | |
| EWSR1 | 2.82 | 2.09 | 0.38 | −0.11 | 0.14 | |
| UBA1 | 2.51 | 0.55 | 0.13 | * | 0.13 | |
| TCERG1 | 3.64 | 2.34 | 0.37 | −0.10 | 0.13 | |
| SUPT16H | 4.01 | 3.66 | 0.23 | 0.04 | 0.13 | |
| HNRNPC | 2.62 | 2.39 | 0.20 | 0.07 | 0.13 | |
| SNRPE | 4.27 | 3.53 | 0.22 | 0.04 | 0.13 | |
| DNMT1 | 2.02 | 1.70 | −0.19 | 0.46 | 0.13 | |
| PUM1 | 1.66 | 2.08 | −0.08 | 0.35 | 0.13 | |
| HNRNPK | 3.64 | 3.13 | 0.10 | 0.17 | 0.13 | |
| FTSJD2 | 3.09 | 2.92 | 0.17 | 0.09 | 0.13 | |
| HNRNPF | 2.25 | 1.66 | −0.02 | 0.28 | 0.13 | |
| SSRP1 | 4.11 | 4.47 | 0.26 | 0.00 | 0.13 | |
| CAPRIN1 | 4.36 | 2.45 | 1.34 | −1.08 | 0.13 | |
| LRWD1 | 2.64 | 2.59 | 0.25 | 0.01 | 0.13 | |
| CFL1 | 3.06 | 2.43 | 0.07 | 0.19 | 0.13 | |
| FAM98B | 3.18 | 3.88 | 0.14 | 0.11 | 0.13 | |
| MINA | 2.12 | 2.27 | −0.08 | 0.34 | 0.13 | |
| ILKAP | 2.54 | 3.22 | 0.33 | −0.07 | 0.13 | |
| PABPN1 | 4.04 | 3.77 | 0.04 | 0.21 | 0.13 | |
| UBAP2 | 1.92 | 2.90 | 0.04 | 0.20 | 0.12 | |
| SRBD1 | 1.29 | 1.02 | 0.37 | −0.13 | 0.12 | |
| SNRPD2 | 3.40 | 3.78 | 0.17 | 0.08 | 0.12 | |
| TOP2A | 2.96 | 2.56 | −0.18 | 0.42 | 0.12 | |
| HIST1H4A | 6.36 | 4.61 | 0.26 | −0.02 | 0.12 | |
| VRK1 | 2.56 | 2.10 | 0.03 | 0.21 | 0.12 | |
| MCTS1 | 2.56 | 2.76 | 0.20 | 0.04 | 0.12 | |
| NUDT21 | 3.15 | 3.65 | 0.04 | 0.20 | 0.12 | |
| MSH6 | 2.41 | 2.51 | 0.06 | 0.18 | 0.12 | |
| PRPF19 | 1.97 | 2.05 | −0.15 | 0.39 | 0.12 | |
| CSDE1 | 2.17 | 2.33 | 0.08 | 0.16 | 0.12 | |
| PRPF38B | 2.96 | 2.44 | 0.26 | −0.03 | 0.12 | |
| CDC73 | 2.60 | 3.07 | −0.14 | 0.37 | 0.12 | |
| RBM14 | 1.89 | 0.82 | 0.17 | 0.06 | 0.11 | |
| HNRNPK | 3.36 | 2.55 | 0.10 | 0.13 | 0.11 | |
| FXR1 | 1.73 | 1.60 | −1.01 | 1.23 | 0.11 | |
| GSR | 2.32 | 2.92 | −0.02 | 0.24 | 0.11 | |
| ZFR | 3.14 | 2.89 | 0.13 | 0.09 | 0.11 | |
| TRIP12 | 1.34 | 0.66 | −0.24 | 0.46 | 0.11 | |
| TRMT61A | 3.82 | 2.78 | 0.29 | −0.07 | 0.11 | |
| MCM7 | 2.52 | 2.23 | 0.10 | 0.12 | 0.11 | |
| SEC23A | 3.17 | 2.56 | −0.01 | 0.22 | 0.11 | |
| SNRNP70 | 3.57 | 3.26 | −0.01 | 0.22 | 0.11 | |
| MCM5 | 2.79 | 2.23 | 0.03 | 0.18 | 0.10 | |
| PRMT3 | 2.05 | 1.71 | 0.07 | 0.13 | 0.10 | |
| RDBP | 2.82 | 2.37 | −0.12 | 0.32 | 0.10 | |
| HNRNPM | 2.44 | 3.18 | 0.19 | 0.01 | 0.10 | |
| PRDX1 | 1.98 | 1.65 | 0.04 | 0.17 | 0.10 | |
| DEK | 2.01 | 2.38 | 0.10 | 0.10 | 0.10 | |
| UBC | 2.42 | 3.82 | −0.12 | 0.32 | 0.10 | |
| HPRT1 | 1.19 | 2.51 | −0.08 | 0.28 | 0.10 | |
| MLLT4 | 2.39 | 3.31 | 0.04 | 0.16 | 0.10 | |
| UBTF | 1.93 | 2.47 | −0.29 | 0.49 | 0.10 | |
| CSNK2B | 2.65 | 2.83 | −0.01 | 0.21 | 0.10 | |
| EEF2 | 2.66 | 2.27 | 0.37 | −0.18 | 0.09 | |
| MSH2 | 1.58 | 1.36 | 0.06 | 0.12 | 0.09 | |
| CGGBP1 | 2.49 | 2.44 | 0.12 | 0.05 | 0.09 | |
| ABCF1 | 2.94 | 2.36 | −0.04 | 0.22 | 0.09 | |
| THYN1 | 2.85 | 2.42 | 0.18 | −0.01 | 0.09 | |
| SRRT | 2.41 | 2.15 | 0.15 | 0.02 | 0.08 | |
| SEC24C | 2.17 | 2.58 | −0.05 | 0.21 | 0.08 | |
| NPM3 | 2.15 | 2.70 | 0.03 | 0.13 | 0.08 | |
| UBAP2L | 1.83 | 2.82 | −0.06 | 0.22 | 0.08 | |
| MAP4 | 2.75 | 1.41 | 0.03 | 0.13 | 0.08 | |
| UBXN1 | 1.91 | 0.62 | 0.04 | 0.12 | 0.08 | |
| SNRNP40 | 2.64 | 2.39 | −0.12 | 0.27 | 0.07 | |
| STRBP | 3.07 | 2.70 | −0.06 | 0.21 | 0.07 | |
| RAD21 | 1.49 | 2.15 | 0.08 | 0.07 | 0.07 | |
| RPRD1A | 1.62 | 2.16 | −0.02 | 0.16 | 0.07 | |
| MRE11A | 2.06 | 1.35 | 0.07 | 0.07 | 0.07 | |
| TK1 | 2.36 | 2.89 | −0.50 | 0.63 | 0.07 | |
| TRIM28 | 1.28 | 1.05 | 0.10 | 0.01 | 0.06 | |
| TUBA1C | 1.45 | 0.42 | 0.93 | −0.83 | 0.05 | |
| PUF60 | 2.61 | 1.71 | 0.07 | 0.03 | 0.05 | |
| STRAP | 1.80 | 1.61 | −0.15 | 0.24 | 0.05 | |
| UHRF1 | 2.24 | 3.68 | −0.45 | 0.54 | 0.04 | |
| ANXA2 | 2.78 | 2.86 | −0.31 | 0.38 | 0.04 | |
| DNAJC9 | 1.85 | 2.56 | −0.02 | 0.09 | 0.04 | |
| MARS | 1.56 | 1.87 | −0.08 | 0.15 | 0.03 | |
| XPC | 3.99 | 2.62 | 0.03 | 0.03 | 0.03 | |
| PAICS | 1.04 | 1.12 | 0.05 | −0.03 | 0.01 | |
| IARS | 2.25 | 2.10 | 0.21 | −0.19 | 0.01 | |
| RAD50 | 2.08 | 2.20 | −0.14 | 0.15 | 0.01 | |
| RBM39 | 0.93 | 2.04 | −0.03 | 0.03 | 0.00 | |
| CD3EAP | 1.86 | 2.30 | 0.01 | −0.02 | 0.00 | |
| CAPZB | 2.61 | 2.30 | 0.31 | −0.31 | 0.00 | |
| FBXO22 | 0.77 | 3.56 | −0.59 | 0.58 | 0.00 | |
| EPRS | 1.98 | 0.46 | 0.63 | −0.64 | −0.01 | |
| RPL38 | 3.21 | 1.23 | 0.29 | −0.32 | −0.02 | |
| DHX36 | 2.83 | 1.95 | −0.24 | 0.19 | −0.02 | |
| DDX49 | 1.77 | 3.17 | −0.49 | 0.43 | −0.03 | |
| SMC1A | 1.83 | 1.31 | −0.01 | −0.05 | −0.03 | |
| CKAP2 | −1.33 | −1.81 | 0.05 | −0.12 | −0.03 | |
| NOSIP | 2.28 | 1.54 | −0.11 | 0.03 | −0.04 | |
| MAP4K5 | 0.87 | 1.69 | −0.72 | 0.63 | −0.04 | |
| GRB2 | 1.36 | 3.13 | −0.45 | 0.36 | −0.05 | |
| NACA | 3.26 | 1.53 | 0.27 | −0.36 | −0.05 | |
| GAPDH | 1.59 | 1.86 | −0.24 | 0.14 | −0.05 | |
| ZRANB2 | 2.59 | 0.71 | −0.02 | −0.09 | −0.06 | |
| GTPBP1 | 1.80 | −0.64 | 0.13 | −0.27 | −0.07 | |
| MAPT | 2.47 | 2.80 | −0.19 | 0.03 | −0.08 | |
| YTHDC2 | 2.87 | 1.74 | −0.07 | −0.10 | −0.08 | |
| SMARCA1 | 3.03 | 0.79 | 0.18 | −0.35 | −0.09 | |
| G6PD | 1.96 | 0.75 | −0.01 | −0.20 | −0.10 | |
| NCBP1 | 2.23 | 1.27 | 0.11 | −0.32 | −0.11 | |
| SMU1 | 2.60 | 1.94 | −0.15 | −0.07 | −0.11 | |
| SHKBP1 | 2.73 | 2.90 | −0.94 | 0.71 | −0.12 | |
| BAZ1B | 0.19 | 1.37 | −0.38 | 0.14 | −0.12 | |
| PRRC2C | 2.21 | 3.32 | −0.44 | 0.19 | −0.13 | |
| SCML2 | 1.28 | 1.39 | −0.85 | 0.59 | −0.13 | |
| RPTOR | 1.58 | 1.86 | −0.81 | 0.54 | −0.14 | |
| EIF4B | 2.50 | 3.26 | 0.03 | −0.32 | −0.15 | |
| CSTF3 | 1.48 | 1.58 | 0.47 | −0.78 | −0.15 | |
| LANCL2 | 1.81 | 1.44 | −0.26 | −0.05 | −0.16 | |
| EIF2S3 | 2.71 | 4.41 | −0.08 | −0.25 | −0.16 | |
| NBN | 1.52 | 1.68 | −0.46 | −0.14 | −0.30 | |
| EEF1B2 | −0.32 | −0.81 | −0.64 | 0.01 | −0.31 | |
| PPP1CA | 0.12 | 1.51 | −1.83 | 1.15 | −0.34 | |
| ERH | 2.39 | 4.11 | −0.99 | 0.21 | −0.39 | |
| EEF1G | −0.28 | −0.67 | −0.77 | −0.03 | −0.40 | |
| DUSP3 | 1.19 | 1.97 | −1.35 | 0.47 | −0.44 | |
| SERBP1 | −0.06 | −0.66 | * | −0.45 | −0.45 | |
| CTNND1 | 2.07 | 0.67 | −0.49 | * | −0.49 | |
| CSRP2 | 1.96 | 1.27 | −0.49 | −0.58 | −0.54 | |
| RPL22 | −0.24 | 3.16 | −1.13 | −0.01 | −0.57 | |
| SMC2 | 0.35 | 0.65 | −1.76 | 0.42 | −0.67 | |
| RUVBL1 | 0.38 | 1.74 | −1.47 | 0.10 | −0.68 | |
| METTL3 | −0.51 | 3.18 | −1.45 | 0.04 | −0.70 | |
| CHEK1 | 0.46 | 0.09 | −0.99 | * | −0.99 | |
| FERMT2 | 1.42 | 3.33 | −0.07 | −1.93 | −1.00 | |
| ORC5 | 2.52 | 1.97 | 0.06 | −2.12 | −1.03 | |
| CAND1 | 3.86 | 3.61 | 1.78 | −3.85 | −1.03 | |
| IK | 1.32 | 2.29 | −3.31 | 0.56 | −1.38 | |
| ING3 | 0.37 | 2.57 | −4.02 | * | −4.02 | |

* indicates that background was higher than signal

While the preferred embodiments of the invention have been illustrated and described, it will be appreciated that various changes can be made therein without departing from the spirit and scope of the invention.

What is claimed is:

1. An affinity reagent that binds to a methylated lysine residue, said affinity reagent comprising one or more engineered 3×MBT domains of one or more L3MBTL1 proteins, wherein each 3×MBT domain consists of residues of a L3MBTL1 protein corresponding to amino acids 190 to 530 of human L3MBTL1 numbered relative to the reference sequence of SEQ ID NO:2.

2. The affinity reagent of claim 2, wherein the affinity reagent comprises contiguous residues 190 to 530 of the amino acid sequence of SEQ ID NO:2.

3. The affinity reagent of claim 2, wherein the affinity reagent comprises
a) a polypeptide comprising the amino acid sequence of SEQ ID NO:29; or
b) a polypeptide comprising an amino acid sequence having at least 95% identity to the amino acid sequence of SEQ ID NO:29, wherein the affinity reagent is capable of binding to a methylated lysine of a protein or peptide.

4. The method of claim 1, wherein the affinity reagent further comprises a tag.

5. The affinity reagent of claim 4, wherein the tag is selected from the group consisting of a FLAG tag, a glutathione S-transferase (GST) tag, a His-tag, a biotin tag, a Strep-tag, a TAP-tag, an S-tag, an SBP-tag, an Arg-tag, a calmodulin-binding peptide tag, a cellulose-binding domain tag, a DsbA tag, a c-myc tag, a HAT-tag, a maltose-binding protein tag, a NusA tag, and a thioredoxin tag.

6. The affinity reagent of claim 5, wherein the tag is a FLAG tag.

7. The affinity reagent of claim 5, wherein the tag is a GST tag.

8. The method of claim 4, wherein the tag is an epitope tag.

9. The affinity reagent of claim 1, wherein the affinity reagent further comprises a detectable label.

10. The affinity reagent of claim 9, wherein the label is selected from the group consisting of a radioactive isotope, a stable non-radioactive heavy isotope, a fluorophore, a chemiluminescer, an enzyme, and a ligand.

11. The affinity reagent of claim 1, further comprising a linker.

12. The affinity reagent of claim 1 immobilized on a solid support.

13. The affinity reagent of claim 12, wherein the solid support is selected from the group consisting of a bead, a particle, a rod, a membrane, a gel, a resin, a column, a chip, a slide, a plate, and a microarray.

14. The affinity reagent of claim 13, wherein the bead is an agarose bead.

15. The affinity reagent of claim 13, wherein the bead is a magnetic bead.

16. The affinity reagent of claim 13, wherein the bead is a microbead.

17. The affinity reagent of claim 13, wherein the plate is a microtiter plate.

18. The affinity reagent of claim 13, wherein the membrane is a nitrocellulose membrane or a polyvinylidene difluoride (PVDF) membrane.

19. A method of isolating a methylated protein or peptide from a mixture, the method comprising binding the affinity reagent of claim 12 to the methylated protein or peptide and performing a pull-down assay to isolate the methylated protein or peptide from the mixture.

20. A method for detecting methylation of a protein or peptide, the method comprising binding the affinity reagent of claim 1 to a methylated protein or peptide and detecting the bound affinity reagent.

21. The method of claim 20, further comprising performing a far-western or a pull-down assay.

22. The method of claim 20, wherein the affinity reagent comprises a detectable label and detecting the bound affinity reagent comprises detecting the label.

23. A method of measuring the activity of a lysine methyltransferase, the method comprising:
a) contacting the lysine methyltransferase with a protein or peptide substrate comprising an unmethylated lysine; and
b) detecting methylation of the lysine in the product of the methyltransferase catalyzed reaction by binding the affinity reagent of claim 1 to the methylated product.

24. The method of claim 23, wherein the affinity reagent comprises a detectable label.

25. The method of claim 23, further comprising identifying substrates of the lysine methyltransferase by contacting the lysine methyltransferase with a protein array comprising candidate protein or peptide substrates.

26. The method of claim 23, wherein the lysine methyltransferase is a mono-methyltransferase.

27. The method of claim 23, wherein the lysine methyltransferase is a di-methyltransferase.

28. A method of isolating a methylated protein or peptide, the method comprising: binding the affinity reagent of claim 8 to the methylated protein or peptide and immunoprecipitating the methylated protein or peptide with an antibody specific for the epitope tag.

29. An affinity chromatography matrix comprising the affinity reagent of claim 1 bound to a solid support.

30. The affinity chromatography matrix of claim 29, wherein the solid support comprises polyether sulfone, agarose, cellulose, a polysaccharide, polytetrafluoroethylene, polysulfone, polyester, polyvinylidene fluoride, polypropylene, poly(tetrafluoroethylene-co-perfluoro(alkyl vinyl ether)), polycarbonate, polyethylene, glass, polyacrylate, polyacrylamide, poly(azolactone), polystyrene, polylactide, ceramic, nylon or metal.

31. The affinity chromatography matrix of claim 29, wherein the solid support is a bead.

32. The affinity chromatography matrix of claim 31, wherein the bead is an agarose, cellulose, or polystyrene bead.

33. The affinity chromatography matrix of claim 29, further comprising a linking group.

34. The matrix of claim 33, wherein the linking group is selected from the group consisting of cyanogen bromide, an aldehyde, an epoxide, and an activated carboxylic acid.

35. The affinity chromatography matrix of claim 29, where the affinity reagent is connected to the solid support through a linker.

36. A method of making the affinity chromatography matrix of claim 33, the method comprising:
a) activating the solid support; and
b) contacting the solid support with the affinity reagent such that the affinity reagent covalently attaches to the solid support.

37. A method of purifying a methylated protein or peptide from a mixture using the affinity chromatography matrix of claim 29, the method comprising:
a) contacting the mixture with the affinity chromatography matrix under a first set of conditions, such that the methylated protein or peptide binds to the immobilized affinity reagent attached to the matrix; and
b) eluting the methylated protein or peptide under a second set of conditions thereby purifying the methylated protein or peptide from the mixture.

38. A kit comprising the affinity agent of claim 1 and instructions for detecting a methylated protein or peptide.

39. The kit of claim 38, further comprising reagents for performing a pull-down assay.

40. The kit of claim 38, further comprising reagents for performing a Far Western.

41. The kit of claim 38, wherein the affinity reagent is immobilized on a solid support.

42. The kit of claim 41, wherein the solid support is selected from the group consisting of a bead, a particle, a rod, a membrane, a gel, a resin, a column, a chip, a slide, a plate, and a microarray.

43. The kit of claim 42, wherein the bead is an agarose bead.

44. The kit of claim 42, wherein the bead is a magnetic bead.

45. The kit of claim 42, wherein the bead is a microbead.

46. The kit of claim 42, wherein the plate is a microtiter plate.

47. The kit of claim 42, wherein the membrane is a nitrocellulose membrane or a polyvinylidene difluoride (PVDF) membrane.

48. A kit comprising the affinity chromatography matrix of claim 29 and instructions for isolating a methylated protein or peptide.

49. The kit of claim 48, further comprising reagents for performing a pull-down assay.

50. The kit of claim 48, further comprising reagents for performing affinity chromatography.

51. A polynucleotide encoding the affinity reagent of claim 1.

52. A recombinant polynucleotide comprising the polynucleotide of claim 51 operably linked to a promoter.

53. The recombinant polynucleotide of claim 52, wherein the recombinant polynucleotide comprises a polynucleotide selected from the group consisting of:
a) a polynucleotide encoding a polypeptide comprising the sequence of SEQ ID NO:29;
b) a polynucleotide encoding a polypeptide comprising a sequence having at least 95% identity to the sequence of SEQ ID NO:29, wherein the encoded polypeptide is capable of binding to a methylated lysine of a protein or peptide;
c) a polynucleotide comprising the contiguous sequence from nucleotide position 677 to nucleotide position 1697 of SEQ ID NO:1; and
d) a polynucleotide comprising a sequence having at least 95% identity to the nucleotide sequence of the polynucleotide of c), wherein the encoded polypeptide is capable of binding to a methylated lysine of a protein or peptide.

54. A host cell comprising the recombinant polynucleotide of claim 52.

55. A method for producing a methyl-lysine affinity reagent, the method comprising the steps of:
a) culturing the host cell of claim 54 under conditions suitable for the expression of the affinity reagent; and
b) recovering the affinity reagent from the host cell culture.

56. A kit comprising the recombinant polynucleotide of claim 52 and instructions for preparing an affinity reagent.

57. The kit of claim 56, further comprising reagents for performing a pull-down assay.

58. The kit of claim 56, further comprising reagents for performing a Far Western.
